SEQUENCE LISTING (49 sequences)

SEQ ID NO: 1 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 2 - DNA, 2824 bp, Homo sapiens
gcggccgcccgcacttcccgcctctggctcgcccgaggacgcgctggcacgcctcccaccccctcactctgactccagctggcgtgcatggtctgcctcgcatcctcacgactcagctccctccctctctcgtgtttttttcctccgccgccccctcattcatccccactgggctccctttccctcaaatgctctggggctctccgcgctttcctgagtccgggctccgaggacccttaggtagtcccggtctcttttaaagctccccggcttccaaagggttgccacgtccctaaaccctgtctccagctcgcatacacacacgcacagacacgcacgttttctgttcctgcgtgacacccgccctcgccgctcggccccgccggtccccgcgcggtgccctcctcccgccacacgggcacgcacgcgcgcgcagggccaagcccgaggcagctcgcccgcagctcgcactcgcaggcgacctgctccagtctccaaagccgatggcatctccgggctctggcttttggtctttcgggtcggaagatggctctggggattccgagaatcccggcacagcgcgagcctggtgccaagtggctcagaagttcacgggcggcatcggaaacaaactgtgcgccctgctctacggagacgccgagaagccggcggagagcggcgggagccaacccccgcgggccgccgcccggaaggccgcctgcgcctgcgaccagaagccctgcagctgctccaaagtggatgtcaactacgcgtttctccatgcaacagacctgctgccggcgtgtgatggagaaaggcccactttggcgtttctgcaagatgttatgaacattttacttcagtatgtggtgaaaagtttcgatagatcaaccaaagtgattgatttccattatcctaatgagcttctccaagaatataattgggaattggcagaccaaccacaaaatttggaggaaattttgatgcattgccaaacaactctaaaatatgcaattaaaacagggcatcctagatacttcaatcaactttctactggtttggatatggttggattagcagcagactggctgacatcaacagcaaatactaacatgttcacctatgaaattgctccagtatttgtgcttttggaatatgtcacactaaagaaaatgagagaaatcattggctggccagggggctctggcgatgggatattttctcccggtggcgccatatctaacatgtatgccatgatgatcgcacgctttaagatgttcccagaagtcaaggagaaaggaatggctgctcttcccaggctcattgccttcacgtctgaacatagtcatttttctctcaagaagggagctgcagccttagggattggaacagacagcgtgattctgattaaatgtgatgagagagggaaaatgattccatctgatcttgaaagaaggattcttgaagccaaacagaaagggtttgttcctttcctcgtgagtgccacagctggaaccaccgtgtacggagcatttgaccccctcttagctgtcgctgacatttgcaaaaagtataagatctggatgcatgtggatgcagcttggggtgggggattactgatgtcccgaaaacacaagtggaaactgagtggcgtggagagggccaactctgtgacgtggaatccacacaagatgatgggagtccctttgcagtgctctgctctcctggttagagaagagggattgatgcagaattgcaaccaaatgcatgcctcctacctctttcagcaagataaacattatgacctgtcctatgacactggagacaaggccttacagtgcggacgccacgttgatgtttttaaactatggctgatgtggagggcaaaggggactaccgggtttgaagcgcatgttgataaatgtttggagttggcagagtatttatacaacatcataaaaaaccgagaaggatatgagatggtgtttgatgggaagcctcagcacacaaatgtctgcttctggtacattcctccaagcttgcgtactctggaagacaatgaagagagaatgagtcgcctctcgaaggtggctccagtgattaaagccagaatgatggagtatggaaccacaatggtcagctaccaacccttgggagacaaggtcaatttcttccgcatggtcatctcaaacccagcggcaactcaccaagacattgacttcctgattgaagaaatagaacgccttggacaagatttataataaccttgctcaccaagctgttccacttctctagagaacatgccctcagctaagccccctactgagaaacttcctttgagaattgtgcgacttcacaaaatgcaaggtgaacaccactttgtctctgagaacagacgttaccaattatggagtgtcaccagctgccaaaatcgtaggtgttggctctgctggtcactggagtagttgctactcttcagaatatggacaaagaaggcacaggtgtaaatatagtagcaggatgaggaacctcaaactgggtatcattttgcacgtgctcttctgttctcaaatgctaaatgcaaacactgtgtatttattagttaggtgtgccaaactaccgttcccaaattggtgtttctgaatgacatcaacattcccccaacattactccattactaaagacagaaaaaaataaaaacataaaatatacaaacatgtggcaacctgttcttcctaccaaatataaacttgtgtatgatccaagtattttatctgtgttgtctctctaaacccaaataaatgtgtaaatgtggacacatctc

SEQ ID NO: 3 - DNA, 1758 bp, Artificial Sequence
  Optimized human GAD65
atggcatctccgggctccggcttttggtccttcgggtcggaagatggctcaggggattccgagaatcccggcacagcgcgggcctggtgtcaagtggctcagaagttcacgggcggcatcggaaacaaactgtgtgccctgctctacggcgacgccgagaagcccgcagagagcggcgggagccaacccccgcgggccgccgcccggaaggccgcctgcgcctgtgaccagaagccctgctcatgcagcaaggtagatgtcaactacgcgtttctccatgccacagatctgctgccggcttgcgacggtgaaaggcccactttggcctttctgcaggatgttatgaacattctgctgcagtacgtggtgaaaagtttcgaccggtcaaccaaagtgatcgactttcactatcctaatgaacttctccaggagtacaattgggagctggctgaccagccacagaacctggaggaaatcttgatgcattgccaaactactctaaaatatgcaattaaaacaggccatcctagatacttcaaccagctttctaccggtttggatatggtggggctggcagccgactggctgacatccaccgcaaataccaacatgttcacctatgagatcgctcctgtcttcgtgcttttggaatacgtcaccctaaagaagatgcgtgaaatcattggctggccaggaggctctggtgatggtatattttctcccggcggcgcgatctctaacatgtatgccatgatgatcgcacgctttaagatgttcccagaagtcaaggagaaaggaatggctgctcttcccaggctcattgccttcacgagtgaacacagtcacttttccctcaagaagggggctgccgccttagggatcggaacagacagcgtgattctgataaagtgcgacgagagagggaaaatgattccatctgatcttgagagaaggattcttgaagccaaacagaaagggtttgtccctttcctcgtgagtgccacagctggaaccaccgtgtacggcgcatttgaccccctcttagctgtcgcggatatatgtaagaagtataagatctggatgcacgtggatgctgcttggggtgggggattactgatgtccaggaaacacaagtggaaactgtctggcgtggagcgcgccaacagcgtgacgtggaatccacacaaaatgatgggagtccctttgcagtgctctgctctcctggttcgagaagagggactgatgcagaattgcaaccaaatgcatgcctcctacctctttcagcaggataaacattatgacctgtcttacgacactggtgacaaggccctgcagtgtgggcgccacgttgatgtattcaagctatggctgatgtggagggcaaaggggactaccggttttgaagcccatgttgacaaatgtctggagttggcagagtatttatacaatatcataaaaaaccgagaaggatatgagatggtgtttgatggcaagcctcagcacacaaatgtctgcttctggtacatccctcccagcctacgtactctggaggacaacgaagagagaatgagtcgcctctcgaaggtggctccagtgattaaagccagaatgatggagtatggaaccacaatggtcagctaccaacccttgggggacaaggtaaatttcttccgcatggtcatctcaaacccagcggcaactcaccaagacattgatttcctgattgaagagatcgagcggctcggccaggatctgtga

SEQ ID NO: 4 - protein, 594 aa, Homo sapiens
MASSTPSSSATSSNAGADPNTTNLRPTTYDTWCGVAHGCTRKLGLKICGFLQRTNSLEEKSRLVSAFKERQSSKNLLSCENSDRDARFRRTETDFSNLFARDLLPAKNGEEQTVQFLLEVVDILLNYVRKTFDRSTKVLDFHHPHQLLEGMEGFNLELSDHPESLEQILVDCRDTLKYGVRTGHPRFFNQLSTGLDIIGLAGEWLTSTANTNMFTYEIAPVFVLMEQITLKKMREIVGWSSKDGDGIFSPGGAISNMYSIMAARYKYFPEVKTKGMAAVPKLVLFTSEQSHYSIKKAGAALGFGTDNVILIKCNERGKIIPADFEAKILEAKQKGYVPFYVNATAGTTVYGAFDPIQEIADICEKYNLWLHVDAAWGGGLLMSRKHRHKLNGIERANSVTWNPHKMMGVLLQCSAILVKEKGILQGCNQMCAGYLFQPDKQYDVSYDTGDKAIQCGRHVDIFKFWLMWKAKGTVGFENQINKCLELAEYLYAKIKNREEFEMVFNGEPEHTNVCFWYIPQSLRGVPDSPQRREKLHKVAPKIKALMMESGTTMVGYQPQGDKANFFRMVISNPAATQSDIDFLIEEIERLGQDL

SEQ ID NO: 5 - DNA, 1784 bp, Homo sapiens
atggcgtctcgaccccatcttcgtccgcaacctcctcgaacgcgggagcggaccccaataccactaacctgcgccccacaacgtacgatacctggtgcggcgtggcccatggatgcaccagaaaactggggctcaagatctgcggcttcttgcaaaggaccaacagcctggaagagaagagtcgccttgtgagtgccttcaaggagaggcaatcctccaagaacctgctttcctgtgaaaacagcgaccgggatgcccgcttccggcgcacagagactgacttctctaatctgtttgctagagatctgcttccggctaagaacggtgaggagcaaaccgtgcaattcctcctggaagtggtggacatactcctcaactatgtccgcaagacatttgatcgctccaccaaggtgctggactttcatcacccacaccagttgctggaaggcatggagggcttcaacttggagctctctgaccaccccgagtccctggagcagatcctggttgactgcagagacaccttgaagtatggggttcgcacaggtcatcctcgatttttcaaccagctctccactggattggatattattggcctagctggagaatggctgacatcaacggccaataccaacatgtttacatatgaaattgcaccagtgtttgtcctcatggaacaaataacacttaagaagatgagagagatagttggatggtcaagtaaagatggtgatgggatattttctcctgggggcgccatatccaacatgtacagcatcatggctgctcgctacaagtacttcccggaagttaagacaaagggcatggcggctgtgcctaaactggtcctcttcacctcagaacagagtcactattccataaagaaagctggggctgcacttggctttggaactgacaatgtgattttgataaagtgcaatgaaagggggaaaataattccagctgattttgaggcaaaaattcttgaagccaaacagaagggatatgttcccttttatgtcaatgcaactgctggcacgactgtttatggagcttttgatccgatacaagagattgcagatatatgtgagaaatataacctttggttgcatgtcgatgctgcctggggaggtgggctgctcatgtccaggaagcaccgccataaactcaacggcatagaaagggccaactcagtcacctggaaccctcacaagatgatgggcgtgctgttgcagtgctctgccattctcgtcaaggaaaagggtatactccaaggatgcaaccagatgtgtgcaggatacctcttccagccagacaagcagtatgatgtctcctacgacaccggggacaaggcaattcagtgtggccgccacgtggatatcttcaagttctggctgatgtggaaagcaaagggcacagtgggatttgaaaaccagatcaacaaatgcctggaactggctgaatacctctatgccaagattaaaaacagagaagaatttgagatggttttcaatggcgagcctgagcacacaaacgtctgtttttggtatattccacaaagcctcaggggtgtgccagacagccctcaacgacgggaaaagctacacaaggtggctccaaaaatcaaagccctgatgatggagtcaggtacgaccatggttggctaccagccccaaggggacaaggccaacttcttccggatggtcatctccaacccagccgctacccagtctgacattgacttcctcattgaggagatagaaagactgggccaggatctgtaa

SEQ ID NO: 6 - protein, 178 aa, rattus norvegicus
MPGSALLCCLLLLAGVKTSKGHSIRGDNNCTHFPVSQTHMLRELRAAFSQVKTFFQKKDQLDNILLTDSLLQDFKGYLGCQALSEMIKFYLVEVMPQAENHGPEIKEHLNSLGEKLKTLWIQLRRCHRFLPCENKSKAVEQVKNDFNKLQDKGVYKAMNEFDIFINCIEAYVTLKMKN

SEQ ID NO: 7 - DNA, 682 bp, rattus norvegicus
catgcctggctcagcactgctatgttgcctgctcttactggctggagtgaagaccagcaaaggccattccatccggggtgacaataactgcacccacttcccagtcagccagacccacatgctccgagagctgagggctgccttcagtcaagtgaagactttctttcaaaagaaggaccagctggacaacatactgctgacagattccttactgcaggactttaagggttacttgggttgccaagccttgtcagaaatgatcaagttttacctggtagaagtgatgccccaggcagagaaccatggcccagaaatcaaggagcatttgaattccctgggagagaagctgaagaccctctggatacagctgcgacgctgtcatcgatttctcccctgtgagaataaaagcaaggcagtggagcaggtgaagaatgattttaataagctccaagacaaaggtgtctacaaggccatgaatgagtttgacatcttcatcaactgcatagaagcctacgtgacactcaaaatgaaaaattgaaccacccggcatctactggactgcaggacataaatagagcttctaaatctgatccagagatcttagctaacgggagcaactccttggaaaacctcgtttgtacctctctccaaaatatttattacctctgatacctcagttccc

SEQ ID NO: 8 - DNA, 537 bp, Artificial Sequence
  Optimized rat IL-10
atgcctggctcagccctgctatgttgccttctcctgctggcgggagtcaagacaagcaagggccattccatccggggagataataactgcacccacttcccagtctctcaaacccacatgttgcgagagctgagggctgccttcagtcaggtgaagacgttcttccagaagaaggaccagctggacaacattctgctgactgacagcctgctgcaggatttcaagggttatttggggtgtcaagccctgtctgaaatgatcaagttttacctggtagaagtgatgccccaggcagagaatcatggccccgagatcaaggagcacctcaactccctgggggagaagctgaagaccctgtggattcagctgaggcgctgccacagatttctcccctgtgaaaacaagagcaaggcagtggagcaggtgaagaacgattttaataagctccaggacaagggcgtctacaaggccatgaacgagttcgacatctttatcaactgcatagaagcttacgttacactcaagatgaagaattga

SEQ ID NO: 9 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - DNA, 1600 bp, Homo sapiens
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 11 - protein, 185 aa, Homo sapiens
MKLWDVVAVCLVLLHTASAFPLPAANMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 12 - DNA, 558 bp, Homo sapiens
atgaagttatgggatgtcgtggctgtctgcctggtgctgctccacaccgcgtccgccttcccgctgcccgccgcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 13 - DNA, 558 bp, Artificial Sequence
  optimized human GDNF
atgaaactttgggacgtggtggctgtctgcctggtgctcctccacaccgccagtgcgtttccgctgcccgccgctaacatgccagaggattatcctgatcagttcgatgatgttatggacttcattcaagccacaatcaagcggctgaaacgatcaccagataaacagatggcagtgcttcctcgccgcgagcgtaatcggcaggctgcagcagccaatcccgagaattcccgaggaaaagggcgcaggggtcagaggggcaagaaccgggggtgtgtcctgactgcaatacatttaaacgtgactgacttgggtctgggctatgagaccaaggaagaactcattttcaggtactgcagcggctcttgcgatgccgcggaaacaacgtacgacaaaatcttgaagaacctctccagaaacagaaggctggtgagtgacaaggtaggacaggcctgttgcagacccatcgcctttgacgacgatctgagctttctggatgacaatctggtttaccacatcctacggaagcattctgctaaaagatgtggatgtatttga

SEQ ID NO: 14 - DNA, 537 bp, Artificial Sequence
  optimized human IL-10
atgcacagctcagcactgctgtgctgcctggtcctgctgacaggggtgagggcaagcccaggccagggaacccaatctgagaacagctgcacccacttccctggcaatctgcctaacatgctgcgcgacctccgagatgccttcagcagagtgaagacttttttccagatgaaggatcagctggacaacctgctgctgaaggagtccctcctggaggactttaagggctacctgggatgccaggccctgtctgagatgatccaattctacctggaagaagttatgccccaggctgagaaccaggacccagacattaaggcccatgtcaactccctgggggaaaatctgaagaccctcaggctgcggctacggcgctgtcaccgttttctgccctgtgagaataagagcaaggctgtggagcaggtgaagaacgccttcaataagctccaggagaagggtatctacaaagcgatgagtgaatttgatatcttcattaattatatagaagcttatatgacaatgaaaatcagaaactga

SEQ ID NO: 15 - DNA, 28 bp, Artificial Sequence
  Forward primer for amplifying CMV promoter (F-JDK)
ttcggccgtcgaggagcttggcccattg

SEQ ID NO: 16 - DNA, 36 bp, Artificial Sequence
  Reverse primer for amplifying CMV promoter (R-JDK)
gacgtcgacctagctagcgaattcggggccgcggag

SEQ ID NO: 17 - DNA, 34 bp, Artificial Sequence
  Forward primer for amplifying SV40pA promoter (F-SV40pA)
ccatcgatcagacatgataagatacattgatgag

SEQ ID NO: 18 - DNA, 43 bp, Artificial Sequence
  Reverse primer for amplifying SV40pA promoter (R-SV40pA)
gacgtcgacgcggccgctaccacatttgtagaggttttacttg

SEQ ID NO: 19 - DNA, 28 bp, Artificial Sequence
  Forward primer for amplifying Kanamycin resistant gene (F-Kan)
aggcgccatgagccatattcaacgggaa

SEQ ID NO: 20 - DNA, 29 bp, Artificial Sequence
  Reverse primer for amplifying Kanamycin resistant gene (R-Kan)
ttcatgattagaaaaactcatcgagcatc

SEQ ID NO: 21 - DNA, 28 bp, Artificial Sequence
  Forward primer for amplifying LITR and CMV (F-ITR)
atggcgcgcccctggccttttgctggcc

SEQ ID NO: 22 - DNA, 28 bp, Artificial Sequence
  Reverse primer for amplifying SV40pA and RITR (R-ITR)
atggatccgctagtaaataccgcatcag

SEQ ID NO: 23 - DNA, 20 bp, Artificial Sequence
  Forward primer for amplifying rIL-10 (F-rIL-10)
ccgctagcgccaccatgcct

SEQ ID NO: 24 - DNA, 39 bp, Artificial Sequence
  Reverse primer for amplifying rIL-10 (R-rIL-10)
gacgtcgacgccatcgatggcttaattaatcaattcttc

SEQ ID NO: 25 - DNA, 40 bp, Artificial Sequence
  Forward primer for WPRE_Pac1_F
ggtggtttaattaaaatcaacctctggattacaaaatttg

SEQ ID NO: 26 - DNA, 30 bp, Artificial Sequence
  Reverse primer for WPRE_modi_Hpa1_R
ggtggtgttaacgacaacaccacggaattg

SEQ ID NO: 27 - DNA, 43 bp, Artificial Sequence
  Forward primer for SV40-CMV-sCAG-bGHpA-Infu-F
cctgcggccggtcgactaccacatttgtagaggttttacttgc

SEQ ID NO: 28 - DNA, 35 bp, Artificial Sequence
  Reverse primer for SV40-CMV-sCAG-bGHpA-Infu-R
aataatcaatgtcgactcgaggagcttggcccatt

SEQ ID NO: 29 - DNA, 42 bp, Artificial Sequence
  nucleotide sequence of Stuffer scramble
gtcgacggtatcgataagcttgatatcgaattcctgcagccc

SEQ ID NO: 30 - DNA, 46 bp, Artificial Sequence
  Forward primer for Stuffer_scramble_F
ctaggtcgacggtatcgataagcttgatatcgaattcctgcagccc

SEQ ID NO: 31 - DNA, 46 bp, Artificial Sequence
  Reverse primer for Stuffer_scramble_R
ctaggggctgcaggaattcgatatcaagcttatcgataccgtcgac

SEQ ID NO: 32 - protein, 585 aa, Canis lupus
MASPGSGFWSFGSEDGSGDPENPSTARAWCQVAQKFTGGIGNKLCALLYGDAEKPAESGGSEPPRATSRKAACACNQKPCSCPKAEVNYAFLHATDLLPACDGERPTLAFLQDVMDILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMVPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYSIIKNREGYEMVFDGKPQHTNVCFWYVPPSLRVLEDNEERMNRLSKVAPVIKARMMEYGTTMVSYQPLGDKVNFFRMVISNPAATHQDIDFLIEEIERLGQDL

SEQ ID NO: 33 - DNA, 1758 bp, Canis lupus
atggcatctccaggctctggcttctggtccttcgggtctgaagatggctccggggatcccgagaaccccagcacagcgagagcctggtgtcaggtggcccagaagttcacgggcggcatcggaaacaagctgtgcgccctgctctacggagatgccgagaagcccgcggagagtggcgggagcgagcccccgcgcgccacctccaggaaggccgcctgcgcttgtaatcagaagccttgcagctgccccaaagcggaggtcaactatgcgtttctacacgcaacagacctgctgccagcctgtgatggagaaaggcccacgttggcgtttctgcaagatgttatggacattttgcttcagtatgttgtgaaaagtttcgatagatcaaccaaagtgattgatttccattaccctaatgagctccttcaagagtataactgggaattggcagaccaaccacaaaatttggaggaaattttgatgcattgccaaacgactctaaaatatgcaattaaaacagggcatcccagatatttcaatcagctttccactggactggatatggttggattagcagcagactggctgacatcaacagcaaacacaaacatgttcacctatgaaattgctccagtatttgtgctcttggaatatgtcacactaaagaaaatgagagaaatcattggctggccgggaggctctggcgatgggatattttctcctggtggcgctatttctaacatgtatgccatgctgatcgcacgctttaagatgttcccagaagtcaaggagaaaggaatggctgcggttcccaggctcattgccttcacatctgagcatagtcacttttctctcaagaagggagctgcagctttggggattggaacagacagcgtgattctgattaaatgtgatgagagggggaaaatggtcccatctgatcttgaaagaaggatccttgaagccaaacaaaaaggatttgttcctttccttgtgagcgccacagctgggaccaccgtgtatggagcattcgaccccctcttagcagttgctgacatttgtaaaaagtacaagatctggatgcatgtggatgctgcttggggtgggggattactgatgtcccggaagcacaaatggaagctgagcggcgtggagagggccaactctgtgacatggaacccacacaagatgatgggcgtccctttacagtgctccgctctcctggttagagaagagggattgatgcagagttgcaaccagatgcatgcctcctacctcttccagcaagataaacactatgacctgtcctatgatactggggataaggccttacagtgtggacgccacgttgatgtttttaaattatggctaatgtggagggcaaagggcaccactgggtttgaagcacatattgataagtgcctggagctggctgagtatttatacagtatcataaaaaaccgagaaggatacgaaatggtgtttgatggaaagcctcagcacacaaatgtctgcttctggtacgtgcctccaagtttgcgtgtcctggaagacaatgaagagagaatgaaccgcctctcaaaggtggccccagtgattaaagcccgaatgatggagtatgggaccacaatggtcagctatcagcccttgggagacaaggtcaatttcttccgcatggttatctcaaatcccgcagcaactcaccaagacatcgacttcctgattgaagaaatagaacgccttggacaagatttataa

SEQ ID NO: 34 - protein, 585 aa, Felis catus
MATPGSGFWSFGSEDGSGDPENPGTARAWCQVAQKFTGGIGNKLCALLYGDSEKPAESGGSQPARATSRKATCACNQKPCSCPKADVNYAFLHATDLLPACDGERPTLAFLQDVMGILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWYVPPSLRVLEDNEERMSRLSKVAPVIKARMMEYGTTMVSYQPLGDKVNFFRMVISNPAATHQDIDFLIEEIERLGQDL

SEQ ID NO: 35 - DNA, 1758 bp, Felis catus
atggcaactccaggctcaggcttttggtccttcgggtctgaagatggctccggggatcccgagaaccccggcacagcgagagcctggtgtcaggtggcccagaagttcacgggcggcatcggaaacaagctgtgcgccctgctctacggggattcagagaagccggcagagagtggagggagccagcccgcgcgggccacctcccggaaggccacctgtgcctgtaaccagaagccttgcagctgccccaaagcggatgtcaactatgcgtttctacacgcaacagacctgctgccagcctgtgatggagaaaggcccactttggcgtttctgcaagatgtaatgggcattttgcttcagtatgtggtgaaaagtttcgacagatcaaccaaagtgattgatttccattaccctaatgagctcctgcaagagtataactgggaattggcagaccaaccacaaaatttggaggaaattttgatgcattgccaaacgactctaaaatatgcaataaaaacagggcatcccaggtacttcaatcaactttccacgggactggatatggttggattagcagcagactggctgacatcaacagcaaacactaatatgttcacctatgaaattgctccagtatttgtgctcttggaatatgtcacactgaaaaaaatgagagaaatcattggctggcctgggggctccggcgatgggatattttctcctggtggcgctatatctaacatgtatgccatgctgattgcacgctttaagatgttcccagaagtcaaggagaaaggaatggctgctgttcccaggctcattgccttcacatccgagcatagtcatttttctctcaagaagggagctgcagctctggggattggaacagacagcgtgattctgattaaatgcgatgagagagggaaaatgatcccatctgatcttgaaagaaggatccttgaagccaaacagaaaggatttgttcctttccttgtgagtgccacagctgggaccactgtgtatggagcatttgaccccctcttggcggtcgctgacatttgcaaaaagtacaagatctggatgcatgtggatgcagcttggggtgggggattactgatgtcccggaaacacaagtggaaactgagcggcgtggagagggccaactctgtgacatggaacccacacaagatgatgggcgtccccttacagtgctctgctctcctggttagagaagaggggttgatgcagagttgcaaccagatgcatgcttcctaccttttccagcaagataaacactacgacctgtcctacgacactggagacaaggccttacagtgtggacgccatgtcgatgtttttaaattatggctaatgtggagggcaaagggcaccactgggtttgaagcacatattgataagtgcttggagctggcagaatatttatacaatatcataaaaaaccgagaaggatatgaaatggtgtttgatggaaagcctcagcacacaaatgtctgcttctggtacgtgcctccaagtttgcgagtcctggaagacaatgaagagagaatgagccgcctctcaaaggtggccccagtgattaaagccagaatgatggagtatgggaccacaatggtcagctatcagcccttgggagacaaggtcaatttcttccgcatggtcatctcaaatcccgcagcaactcaccaagacattgacttcctgattgaagaaatagaacgccttggacaagatttataa

SEQ ID NO: 36 - protein, 585 aa, Equus caballus
MASPGSGFWSFGSEDGSGDPENPGTARAWCQVAQKFTGGIGNKLCALLYGDAEKAAESGGSEPPRATSRKAACSCNQKPCSCSKADVNYAFLHATDLLPACDGERPTLAFLQDVMDILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMLIARFKMFPEVKEKGMAAVPRLIAFTSEHSHFSLKKGAAALGIGTDSVILIRCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQSCNQMHASYLFQQDKHYDLSYDTGDKALQCGRHVDVFKLWLMWRAKGTTGFEAHIDKCLELAEYLYNIIKNREGYEMVFDGKPQHTNVCFWYVPPSLRVLEDNEERMSRLSKVAPVIKARMMEYGTTMVSYQPLGDKVNFFRMVISNPAATHQDIDFLIEEIERLGQDL

SEQ ID NO: 37 - DNA, 1758 bp, Equus caballus
atggcatctcccggctccggcttttggtcctttgggtctgaagatggctccggggatcccgagaaccctggcacagcgagagcctggtgtcaggtggcccagaagttcaccggcggcatcggaaacaagctatgcgccctgctctacggagacgccgagaaggcggcggagagcggcgggagcgagcccccgcgggccacctcccggaaggccgcctgctcctgcaaccagaagccctgcagctgctccaaagccgatgtcaactatgcgtttctacacgcaacagacttgctgccagcttgtgacggagaaagacccactttggcgtttctgcaagatgttatggacattttgcttcagtatgtggtgaaaagtttcgatagatcaaccaaagtgattgacttccattaccctaatgagctccttcaagagtataattgggaattggcagaccaaccacaaaatctggaggaaattttgatgcattgccaaacaactttaaaatatgcaattaaaacagggcatcctagatatttcaatcaactttccactggactggatatggttggattagcagcagactggctgacatcaacagcaaacaccaacatgttcacctatgaaattgctccagtattcgtgcttttggaatatgtcacattaaagaaaatgagagaaatcattggctggccaggaggctctggcgatggaatattttctcctggtggcgccatatctaacatgtatgccatgctgattgcacgctttaagatgttcccagaagtcaaggagaaaggaatggccgctgttcccaggctcattgccttcacgtctgagcatagtcatttttctctcaagaagggagctgcagccttggggattggaacagacagcgtaattctgattagatgtgatgagagggggaaaatgatcccatcggatcttgaaagaagaatccttgaagccaaacaaaaaggatttgtcccttttcttgtgagtgccacggctgggaccaccgtgtatggagcattcgatcccctcttagctgtcgctgacatttgcaaaaagtacaagatctggatgcatgtggatgcagcttggggcgggggattactgatgtcccggaaacacaagtggaaactgagtggcgtggagagggccaactctgtgacatggaatccacacaagatgatgggtgtccctttgcagtgctctgctctcctggttagagaagagggattgatgcagagttgcaaccagatgcatgcctcctacctctttcagcaagataaacactatgacctgtcctatgacactggagacaaggccttgcagtgcggacgccacgtggatgtttttaagttatggctcatgtggagggcaaagggaacaactgggtttgaagcacatattgataagtgtttggagttggcggagtatttatacaatatcataaaaaaccgagaaggatatgaaatggtgtttgacggaaagcctcagcacaccaatgtctgcttctggtatgtacctccgagtctgcgtgttctagaagacaatgaagagagaatgagccgcctctcaaaggtggccccggtgattaaagccagaatgatggagtatgggaccacaatggtcagctaccagcccttgggagacaaggtcaatttcttccgcatggtcatctcaaatcccgcagcaactcaccaagacattgacttcctgattgaagaaatagaacgccttggacaagatttataa

SEQ ID NO: 38 - protein, 181 aa, Canis lupus
MHGSALLCCCLVLLAGVGASRHQSTLLEDDCTHFPASLPHMLRELRAAFGRVKIFFQMKDKLDNILLTGSLLEDFKSYLGCQALSEMIQFYLEEVMPRAENHDPDIKNHVNSLGEKLKTLRLRLRLRRCHRFLPCENKSKAVEQVKSAFSKLQEKGVYKAMSEFDIFINYIETYMTMRMKI

SEQ ID NO: 39 - DNA, 546 bp, Canis lupus
atgcatggctcagcactgctctgttgctgcctggtcctcctggccggggtgggagccagccgacaccagagcaccctacttgaggacgactgcacccacttcccagccagcctgccccacatgctccgagagctccgagctgccttcgggagggtgaagatcttctttcaaatgaaggacaagctggacaacatactgctgaccgggtccctgctggaggactttaagagttacctgggttgccaagccctgtcggagatgatccagttttacttggaggaggtgatgccccgggctgagaaccacgacccagacatcaagaaccacgtgaactccctgggagagaagctcaagaccctcaggctgagactgaggctgcgacgctgtcaccgatttcttccctgtgagaataagagcaaggcggtggagcaggtgaagagcgcatttagtaagctccaggagaaaggtgtctacaaagccatgagtgagtttgacatcttcatcaactacatagaaacctacatgacaatgaggatgaaaatctga

SEQ ID NO: 40 - protein, 178 aa, Felis catus
MHSSALLCFLVFLAGVGASRHQSTLSEDNCTHFSVSLPHMLRELRAAFGKVKTFFQTKDELHSILLTRSLLEDFKGYLGCQALSEMIQFYLEEVMPQAENEDPDIKQHVNSLGEKLKTLRLRLRRCHRFLPCENKSKVVEQVKSTFSKLQEKGVYKAMGEFDIFINYIEAYMTMKMKI

SEQ ID NO: 41 - DNA, 537 bp, Felis catus
atgcacagctcagcacttctgtgtttcctggtcttcctggccggggtaggagccagccgacaccagagcaccctgtctgaggacaactgcacccacttctcagtcagcctgccccacatgctccgagagctccgagctgccttcggcaaggtgaagactttctttcaaaccaaggacgagctgcacagcatattgttgaccaggtccttgctggaggactttaagggttacctgggttgccaagccttgtccgagatgatccagttttatttggaggaggtgatgccccaggctgagaacgaggacccagacatcaaacagcacgtgaactccctgggagaaaagctgaagaccctccggctgagactgcggcgctgtcatcgatttctgccctgtgaaaacaagagcaaggtggtggagcaggtgaagagtacctttagtaagctccaagagaaaggtgtctacaaagccatgggtgagtttgacatcttcatcaactacatagaagcttacatgacaatgaagatgaaaatctga

SEQ ID NO: 42 - protein, 178 aa, Equus caballus
MHSSALLCYLVFLAGVGASRDRGTQSENSCTHFPTSLPHMLHELRAAFSRVKTFFQMKDQLDNMLLNGSLLEDFKGYLGCQALSEMIQFYLEEVMPQAENHGPDIKEHVNSLGEKLKTLRVRLRRCHRFLPCENKSKAVEQVKSAFSKLQEKGVYKAMSEFDIFINYIEAYMTTKMKN

SEQ ID NO: 43 - DNA, 537 bp, Equus caballus
atgcacagctcagcactgctatgttacctggtcttcctggccggggtgggagccagccgagaccggggcacccagtctgagaacagctgcacccacttcccaaccagcctgccccacatgctccatgagctccgagccgccttcagcagggtgaagactttctttcaaatgaaggaccagctggacaacatgttgttgaacgggtccctgctggaggactttaagggttacctgggttgccaagccttgtcggagatgatccagttttacctggaggaggtgatgccccaggctgagaaccacggcccagacatcaaggagcacgtgaactccctgggggaaaagctgaagaccctccgagtgaggctgcggcgctgtcatcgatttctgccctgtgaaaataagagcaaggcagtggagcaggtgaagagtgccttcagtaagctccaagagaaaggtgtctacaaagccatgagtgagtttgacatcttcatcaactacatagaagcctatatgacaacgaagatgaaaaactga

SEQ ID NO: 44 - protein, 185 aa, Canis lupus
MKLWDVVAVCLVLLHTASALPLPAANVPEDYSDQFDDVMDFIQATIRRLKRSPEKQMAVPARRERNRQAAAAGPEHSRGKGRRGPRGRNRGCVLTAIHLNVTDLGLGYETKEELIFRYCSGSCDAAETMYDKILKNLSKSRRLASDKAGQACCRPIAYDDDLSFLDDNLVYHILRKHSAKRCGCI

SEQ ID NO: 45 - DNA, 558 bp, Canis lupus
atgaagttatgggatgtcgtggctgtctgcctggtgctgctccacaccgcgtccgccctcccgctgcccgccgcaaacgtgccggaggactattctgatcagtttgatgacgtcatggattttattcaggccaccatcagaaggctgaaaaggtcacccgagaaacaaatggccgtgccagcgagacgagagcggaatcgtcaggccgcggccgccggcccggaacattccagggggaaggggcggcgaggcccgaggggcagaaaccggggttgtgtcttgactgcgatacatttaaacgtcactgacctgggcttgggctacgaaaccaaggaggaactgatttttaggtactgcagcggctcctgcgacgcggccgagaccatgtacgacaaaatattaaaaaacttatccaaaagtagaaggctggcgagtgacaaagcagggcaggcttgctgcagacccatcgcctacgatgacgacctgtcgtttttagatgacaacctggtttaccatattctaagaaagcattccgctaaaaggtgtggatgtatctga

SEQ ID NO: 46 - protein, 211 aa, Felis catus
MKLWDVVAVCLVLLHTASAFPLPAGKRPPEAPAEDRSLGRRRAPFALSSDSNMPEDYPDQFDDVMDFIQATIRRLKRSPEKQMALPPRRERNRQAAAANPENSRGKGRRGQRGRNRGCVLTAIHLNVTDLGLGYETKEELIFRYCSGSCDAAETMYDKILKNLSKNRRLVSDKVGQACCRPIAYDDDLSFLDDNLVYHILRKHSAKRCGCI

SEQ ID NO: 47 - DNA, 636 bp, Felis catus
atgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagtttgacgacgtcatggattttattcaagctaccatcagaagactgaaaaggtcacccgagaaacaaatggccttgccgcctagaagagagcggaatcggcaggcggcggccgccaacccggagaattccagagggaaaggtcggcgaggccagaggggcagaaatcggggttgtgtcttaactgcgatacatttgaacgtcaccgacctgggtttgggctacgaaaccaaggaggaactgatttttaggtactgcagcggctcctgtgatgcagctgagacaatgtacgacaaaatattaaaaaacttatccaaaaacagaaggctggtgagtgacaaagtcgggcaggcatgttgcagacccatcgcctatgacgacgacctgtcgtttttagatgacaacctggtttaccatattctaagaaagcattccgctaaaaggtgtggatgtatctga

SEQ ID NO: 48 - protein, 185 aa, Equus caballus
MKLWDVVAVCLVLLHTASAFPLPAANMPEDYPDQFDDVMDFIQATIKRLKRSPDKQMAVLPRRERNRQAAAANPENSRRKGQRGQRGKNRGCVLTAIHLNVTDLGLGYETKEELIFRYCSGSCEAAETMYDKILKNLSKNRRLVSDKVGQACCRPIAFDDDLSFLDDNLVYHILRKHSAKRCGCI

SEQ ID NO: 49 - DNA, 558 bp, Equus caballus
atgaagttatgggatgtcgtggctgtctgcctggtgctgctccacaccgcgtccgccttcccgctgcccgccgcaaatatgccagaggattatcctgatcagtttgatgatgtcatggattttattcaagccaccattaaaagactgaaaaggtcaccagataaacaaatggcagtgcttcctagaagagagcggaatcggcaggctgcagctgccaacccggagaattccagaaggaaaggtcagcgaggccagaggggcaaaaaccggggttgtgtcttaaccgcgatacatttaaatgtcactgacttgggtttgggctacgaaaccaaggaggaactgatttttaggtactgcagtggctcctgcgaggcagccgagacaatgtacgacaaaatattaaaaaacttatccaaaaatagaaggctggtgagtgacaaagtagggcaggcatgttgcagacccatcgccttcgatgacgacctgtcatttttagatgataacttggtttaccatattctaagaaagcattccgctaaaaggtgtggatgtatctga

(19) United States
(12) Patent Application Publication
(10) Pub. No.: US 2019/0255152 A1
(43) Pub. Date: Aug. 22, 2019

(54) COMPOSITION FOR ALLEVIATING OR TREATING PAIN

(71) Applicant: KOLON LIFE SCIENCE, INC., Seoul (KR)

(72) Inventors: Sujeong KIM, Seoul (KR); Heonsik CHOI, Seoul (KR); Yejin KWON, Seoul (KR); Minjung KIM, Seoul (KR); Minju KIM, Seoul (KR); Daewook KIM, Yongin-si (KR); Jangjoon PARK, Seoul (KR); Jongho CHO, Seoul (KR); Soondong LEE, Gwangmyeong-si (KR); Joonsung KIM, Suwon-si (KR); Yeomoon SIM, Seoul (KR)

(73) Assignee: KOLON LIFE SCIENCE, INC., Seoul (KR)

(21) Appl. No.: 16/346,170

(22) PCT Filed: Oct. 31, 2017
(86) PCT No.: PCT/KR2017/012136

§ 371 Date: Apr. 30, 2019

(30) Foreign Application Priority Data
Oct. 31, 2016 (KR) 10-2016-0143519

(51) Int. Cl. A61K 38/20; A61K 9/00; A61K 38/18; A61K 38/51; A61P 25/04; C12N 15/86

(52) U.S. Cl. CPC A61K 38/2066; A61K 9/0019; A61K 38/185; A61K 48/00; A61P 25/04; C12Y 401/01015; C12N 15/86; A61K 38/51

(57) ABSTRACT

The present invention relates to a composition for alleviating or treating pain. A pharmaceutical composition of the present invention contains two or more selected from the group consisting of GAD, IL-10, and a gene encoding GDNF. The pharmaceutical composition of the present invention exhibits an excellent analgesic effect at a dosage lower than that of individual administration since genes are coadministered, and thus conventional side effects and toxicity can be reduced. Therefore, the pharmaceutical composition of the present invention can be useful in alleviating or treating pain.

### TECHNICAL FIELD

The present invention relates to a composition for alleviating or treating pain and a method for alleviating or treating pain using the same.

### BACKGROUND ART

Pain means an experience of actual or potential tissue damage or unpleasant sensations and feelings associated with such damage. Pain protects parts of the body that have been damaged during the healing of the damaged tissues from the damaged situation and provides motivation to avoid similar experiences in the future. Most pain is alleviated slowly when the causal stimulus is removed, but sometimes pain persists even though the tissues have been healed as the stimulus has disappeared and the damage has clearly healed, or pain occurs in a state without any irritation, damage or disease.

For the treatment of pain, mainly, narcotic analgesics such as morphine, which is an opioid alkaloid, or non-narcotic analgesics such as non-steroidal anti-inflammatory drugs (NSAIDs) having the ingredient of acetylsalicylic acid, ibuprofen, or acetaminophen are widely used.

Narcotic analgesics have the advantage of showing the dose-response and high efficacy, but they can lead to nervous system side effects and if used for a long period, they can lead to the resistance and physical dependence, and pain may worsen.

If aspirin, a non-narcotic analgesic having acetylsalicylic acid as a main ingredient, is used for an analgesic purpose, it should be administered at a high dose of at least 500 mg. However, aspirin is a non-steroidal anti-inflammatory analgesic that blocks the enzyme (COX-1) that promotes the production of prostaglandins, which protect the stomach, thereby preventing gastric mucosa formation. Therefore, the stomach may be easily damaged by gastric acid and gastrointestinal bleeding may occur. In addition, ibuprofen is also a non-steroidal anti-inflammatory analgesic, which can cause gastric disturbances. Also, in the case of analgesics having acetaminophen as a main ingredient, such as Tylenol, acetaminophen is mostly metabolized in the liver, and liver damage may be induced.

Even if the above analgesics are effective at an early stage, they often become ineffective due to resistance when used for a long period. Specifically, in the case of neuropathic pain, there is a problem that the pain is non-responsive to the maximum dose of a nonsteroidal anti-inflammatory agent, and thus, it is administered at a high dose for a short period.

Recently, new therapeutic agents for neuropathic pain have been developed, but still have side effects. For example, sodium channel blockers are mostly in the form of small molecules and show low selectivity for isoform proteins. In addition, they show side effects such as cardiac toxicity and movement disorder.

Therefore, there is an imperative need to develop a new analgesic for neuropathic pain which is excellent in analgesic efficacy while reducing side effects.

### DISCLOSURE OF INVENTION

### Technical Problem

Accordingly, the present inventors have endeavored to develop a new analgesic for neuropathic pain exhibiting an excellent analgesic efficacy even at a low dosage. As a result, the present inventors have found that when a combination of two or more of glutamate decarboxylase, an anti-inflammatory cytokine, and a glial cell-derived neurotrophic factor is used, pain can be significantly alleviated or treated as compared with an individual use, and have completed the present invention.

### Solution to Problem

The present invention provides a pharmaceutical composition for alleviating or treating pain comprising two or more selected from the group consisting of a gene encoding glutamate decarboxylase (GAD), a gene encoding interleukin-10 (IL-10), and a gene encoding a glial cell-derived neurotrophic factor (GDNF).

In addition, the present invention provides a method for alleviating or treating pain, comprising administering the pharmaceutical composition according to the present invention.

### Advantageous Effects of Invention

A pharmaceutical composition of the present invention comprises two or more selected from the group consisting of genes encoding GAD, IL-10, and GDNF. Therefore, the pharmaceutical composition of the present invention exhibits an excellent analgesic efficacy at a dosage lower than that of individual administration since genes are co-administered, and thus conventional side effects and toxicity can be reduced. Therefore, the pharmaceutical composition of the present invention can be useful in alleviating or treating pain.

### BRIEF DESCRIPTION OF DRAWINGS

FIG. 1 shows the schematic diagram of the plasmids pAAV-GAD65 and pAAV-GAD65-modi used for the construction of the recombinant adeno-associated virus:

(a) shows the schematic diagram of pAAV-GAD65, and (b) shows the schematic diagram of pAAV-GAD65-modi.

FIG. 2 shows the schematic diagram of the plasmid pAAV-IL-10 used for the construction of the recombinant adeno-associated virus.

FIG. 3 shows the schematic diagram of the plasmid pAAV-GDNF used for the construction of the recombinant adeno-associated virus.

FIG. 4 is a schematic diagram showing the pAAV-GDNF-IL-10 plasmid.

FIG. 5 shows the expression of each introduced gene by the pAAV-GAD65, pAAV-IL-10, or pAAV-GDNF plasmid:

(a) shows the expression of GAD65 by pAAV-GAD65 plasmid; (b) shows the expression of IL-10 by pAAV-IL-10 plasmid; and (c) shows the expression of GDNF by the pAAV-GDNF plasmid.

FIG. 6 shows the expression of GDNF gene and IL-10 gene by the pAAV-GDNF-IL-10 plasmid:

(a) shows the expression of IL-10 by pAAV-GDNF-IL-10 plasmid; and (b) shows the expression of GDNF by pAAV-GDNF-IL-10 plasmid.

FIG. 7 shows the results of Western blot showing expression of each protein after treatment of 293T or HeLa cells with each recombinant adeno-associated virus after construction of the recombinant adeno-associated viruses into which GAD65 gene, IL-10 gene and GDNF gene were introduced, respectively:

(a) shows the expression of GAD65 after treatment of 293T or HeLa cells with the recombinant adeno-associated virus AAV-GAD65; (b) shows the expression of GAD65 after treatment of 293T or HeLa cells with the recombinant adeno-associated virus AAV-GAD65-modi; (c) shows the expression of IL-10 after treatment of 293T or HeLa cells with the recombinant adeno-associated virus AAV-IL-10; and (d) shows the expression of GDNF after treatment of 293T or HeLa cells with the recombinant adeno-associated virus AAV-GDNF.

FIG. 8 shows the levels of GABA expression measured by ELISA after treatment of 293T or HeLa cells with the recombinant adeno-associated virus AAV-GAD65 or AAV-GAD65-modi:

(a) is a graph showing the level of GABA expression after 293T or HeLa cells were treated with the recombinant adeno-associated virus AAV-GAD65; and (b) is a graph showing the level of GABA expression after 293T or HeLa cells were treated with the recombinant adeno-associated virus AAV-GAD65-modi.

FIG. 9 is a graph showing the results of comparing the pain alleviating efficacies between individual administration of AAV-GAD65, AAV-IL-10, or AAV-GDNF virus and co-administration of AAV-GAD65 and AAV-GDNF viruses, or AAV-IL-10 and AAV-GDNF viruses.

FIG. 10 is a graph showing the results of comparing the pain alleviating efficacies between individual administration of AAV-GAD65, AAV-IL-10, or AAV-GDNF virus and co-administration of AAV-GAD65 and AAV-IL-10 viruses.

FIG. 11 is a graph showing the results of comparing the pain alleviating efficacies between co-administration of AAV-GAD65 and AAV-GDNF viruses, or AAV-IL-10 and AAV-GDNF viruses and co-administration of all of the AAV-GAD65, AAV-IL-10 and AAV-GDNF viruses.

FIG. 12 is a graph showing the results of comparing the pain alleviating efficacies between individual administration of pAAV-GAD65, pAAV-IL-10, or pAAV-GDNF plasmid and co-administration of pAAV-GAD65 and pAAV-GDNF plasmids, or pAAV-IL-10 and pAAV-GDNF plasmids.

FIG. 13 is a graph showing the results of comparing the pain alleviating efficacies between individual administration of pAAV-GAD65, pAAV-IL-10, or pAAV-GDNF plasmid and co-administration of all of the pAAV-GAD65, pAAV-IL-10 and pAAV-GDNF plasmids.

FIG. 14 is a graph showing the results of comparing the pain alleviating efficacies between co-administration of pAAV-GAD65 and pAAV-GDNF plasmids, or pAAV-IL-10 and pAAV-GDNF plasmids and co-administration of all of the pAAV-GAD65, pAAV-IL-10 and pAAV-GDNF plasmids.

FIG. 15 is a graph showing the results of comparing the pain alleviating efficacies between co-administration of AAV-GAD65-modi and AAV-GDNF-IL-10 viruses and co-administration of all of the AAV-GAD65, AAV-IL-10 and AAV-GDNF viruses.

### BEST MODE FOR CARRYING OUT THE INVENTION

Hereinafter, the present invention will be described in detail.

The present invention provides a pharmaceutical composition for alleviating or treating pain comprising two or more selected from the group consisting of a gene encoding glutamate decarboxylase (GAD), a gene encoding interleukin-10 (IL-10), and a gene encoding a glial cell-derived neurotrophic factor (GDNF).

In one embodiment, the combination of two or more may be GAD and IL-10, GAD and GDNF, IL-10 and GDNF, or GAD, IL-10 and GDNF.

Two or more genes selected from the group consisting of a gene encoding GAD, a gene encoding IL-10, and a gene encoding GDNF may be in a form of being contained in a carrier. Herein, the carrier may be a viral vector, or a non-viral vector such as a plasmid, a liposome, etc. In addition, the genes may be in a form in which some of the genes are contained in a viral vector and the remaining genes are contained in a non-viral vector.

In one embodiment, the genes may be in a form in which GAD is contained in a viral vector, and IL-10 is contained in a non-viral vector. In addition, the genes may be in a form in which GAD is contained in a viral vector, and GDNF is contained in a non-viral vector. Further, the genes may be in a form in which IL-10 is contained in a viral vector, and GDNF is contained in a non-viral vector. In addition, the genes may be in a form in which GAD is contained in a viral vector, and IL-10 and GDNF are contained in a non-viral vector. In addition, the genes may be in a form in which GAD and IL-10 are contained in a viral vector, and GDNF is contained in a non-viral vector. In addition, the genes may be in a form in which GAD and GDNF are contained in a viral vector, and IL-10 is contained a non-viral vector. In addition, the genes may be in a form in which IL-10 is contained in a viral vector, and GAD and GDNF are contained in a non-viral vector. In addition, the genes may be in a form in which IL-10 and GDNF are contained in a viral vector, and GAD is contained in a non-viral vector. Also, the genes may be in a form in which GDNF is contained in a viral vector, and GAD and IL-10 are contained in a non-viral vector.

In addition, the gene may be in a form of being operably contained in a vector. Specifically, the gene may be in a form of being operably contained in a viral vector or a non-viral vector.

The viral vector may be at least one selected from the group consisting of adenovirus, adeno-associated virus (AAV), herpes simplex virus, lentivirus, retrovirus, cytomegalovirus, baculovirus, poxvirus, etc. Specifically, the viral vector may be adeno-associated virus.

In one embodiment, the gene encoding GAD may be operably contained in a carrier 1 (e.g., a first vector), and the gene encoding IL-10 may be operably contained in a carrier 2 (e.g., a second vector), and the gene encoding GDNF may be operably contained in a carrier 3 (e.g., a third vector). In addition, one carrier may contain two or more genes.

The non-viral vector may be at least one selected from the group consisting of a plasmid, a liposome, a cationic polymer, a micelle, an emulsion, and solid lipid nanoparticles.

The term “plasmid” as used herein refers to a circular DNA fragment existing separately outside the chromosome of bacteria. Plasmids have no genes essential for the survival of bacteria, but contain genes essential for resistance to certain antibiotics and for interbacterial gene exchange. In addition, plasmids can grow independently of chromosomes and contain selectable markers.

The term “liposome” as used herein refers to a small vesicle produced by forming a bilayer due to the hydrophilic portion and the hydrophobic portion when a molecule having both a hydrophobic portion and a hydrophilic portion in a molecule, such as a phospholipid, is suspended in an aqueous solution. Liposome is isolated from the outer membrane by a membrane composed of a lipid bilayer, and liposomes containing DNA, mRNA, etc., can be used as mediators of genetic information.

The term “cationic polymer” as used herein refers to a cationic lipid or a polymer compound which is a substance that forms a complex by an ionic bond with anionic DNA and delivers the DNA into a cell.

The term “micelle” as used herein refers to a thermodynamically stable colloidal aggregate formed from the molecules consisting of a polar group and a nonpolar hydrophobic group, such as surfactants and lipid molecules, through association by a van der Waals force or the like in a solution. In addition, micelles containing DNA, mRNA and the like can be used as mediators of genetic information.

The term “emulsion” as used herein means that, when two solutions of different phases are mixed, one liquid forms fine particles and is dispersed in another liquid. DNA, mRNA and the like may be contained in the center of the emulsion particle to be used as mediators of genetic information.

As used herein, the term “solid lipid nanoparticle” refers to a preparation of a form in which a drug is contained in a nano-sized microparticle made of a solid lipid instead of a liquid lipid.

A carrier 1 (e.g., a first vector) comprising any one gene selected from the group consisting of GAD, IL-10, and GDNF, and a carrier 2 (e.g., a second vector) comprising any one gene selected from the remaining gene group not included in the carrier 1 according to the present invention may have a virus titer-based mixing ratio per unit volume of 1: 1 to 100 or 1 to 100: 1. Specifically, the virus titer-based mixing ratio per unit volume of the carrier 1 and the carrier 2 may be 1: 1 to 10 or 1 to 10: 1.

A carrier 1 (e.g., a first vector) comprising a gene encoding GAD, a carrier 2 (e.g., a second vector) comprising a gene encoding IL-10 and a carrier 3 (e.g., a third vector) comprising a gene encoding GDNF according to the present invention may have a virus titer-based mixing ratio per unit volume of 1: 0.1 to 10: 0.1 to 10.

As used herein, the term “operably” means that an introduced gene is linked to a regulatory sequence in such a way that expression can take place in a host cell. The regulatory sequence is a DNA sequence that regulates the expression of the gene, and may include other regulatory elements such as promoters and enhancers or polyadenylation. In addition, the regulatory sequence provides a site for binding of a transcription factor that controls the expression of the introduced gene, and can influence the complex structure with the transcription factor to determine the function of the transcription factor.

The term “GAD” as used herein refers to an enzyme that decarboxylates glutamate to produce GABA (gamma-aminobutyric acid). The GAD may be GAD65 or GAD67. Specifically, GAD65 may be derived from a human, a rat, a dog, a cat, or a horse, but is not limited thereto. The gene encoding GAD may be the nucleotide sequence encoding the amino acid sequence represented by SEQ ID NO: 1, 4, 32, 34, or 36.

In addition, the nucleotide sequence encoding the amino acid sequence represented by SEQ ID NO: 1 may be the DNA sequence represented by SEQ ID NO: 2 or 3, and may be the mRNA sequence shown in NCBI Reference Sequence: NM_000818.2. In addition, the nucleotide sequence encoding the amino acid sequence represented by SEQ ID NO: 4 may be the nucleotide sequence which was codon-optimized to be suitable for the DNA sequence represented by SEQ ID NO: 5 or the gene encoding the amino acid sequence represented by SEQ ID NO: 4, which may be the mRNA sequence shown in NCBI Reference Sequence: NM_000817.2. In addition, the nucleotide sequence encoding the amino acid sequence represented by SEQ ID NO: 32 may be the DNA sequence represented by SEQ ID NO: 33. The nucleotide sequence encoding the amino acid sequence represented by SEQ ID NO: 34 may be the DNA sequence represented by SEQ ID NO: 35, and the nucleotide sequence encoding the amino acid sequence represented by SEQ ID NO: 36 may be the DNA sequence represented by SEQ ID NO: 37.

In addition, the gene encoding GAD may be a nucleotide sequence encoding a GAD variant which can retain GAD activity and produce GABA. The GAD variant includes all sequences which retain GAD's characteristics of producing GABA. Although not limited to any one sequence, the nucleotide sequence encoding the GAD variant may be, preferably, a nucleotide sequence encoding an amino acid sequence having a sequence homology of at least 60% or more, 70% or more, 80% or more, or 90% or more, and to the GAD's amino acid sequence described above, and most preferably, may be a nucleotide sequence encoding an amino acid sequence having a sequence homology of 95% or more.

In addition, the nucleotide sequence encoding the GAD variant may be a nucleotide sequence having a sequence homology of at least 60% or more, 70% or more, 80% or more, or 90% or more to the GAD nucleotide sequence described above, and most preferably, may be a nucleotide sequence having a sequence homology of 95% or more.

The “% of sequence homology” is determined by comparing the comparison regions in a state in which two sequences are optimally aligned. In addition, some of the nucleotide sequences in the comparison regions may include additions or deletions (i.e., gaps) relative to the reference sequence (without addition or deletion) for the optimal alignment of the two sequences.

The term “IL-10” as used herein refers to an anti-inflammatory cytokine belonging to the class II cytokine (Renauld, Nat Rev Immunol, 2003). The IL-10 is in a form of a homodimer consisting of two subunits each of which has the length of 178 amino acids. It is also known as the cytokine synthesis inhibitory factor (CSIF) in humans. IL-10 serves the function of inhibiting the activity of NK (natural killer) cells in the immune response, and forms a complex with an IL-10 receptor to be involved in signal transduction. IL-10 may be a protein derived from a human, a rat, a dog, a cat, or a horse, but is not limited thereto. The gene encoding IL-10 may be the nucleotide sequence encoding the amino acid sequence represented by SEQ ID NO: 6, 9, 38, 40, or 42.

Specifically, the nucleotide sequence encoding the amino acid sequence represented by SEQ ID NO: 6 may be the DNA sequence represented by SEQ ID NO: 7 or 8, and may be the mRNA sequence shown in NCBI Reference Sequence: NM_012854.2. In addition, the nucleotide sequence encoding the amino acid sequence represented by SEQ ID NO: 9 may be the DNA sequence represented by SEQ ID NO: 10 or 14, and may be the mRNA sequence shown in NCBI Reference Sequence: NM_000572.2. The nucleotide sequence encoding the amino acid sequence represented by SEQ ID NO: 38 may be the DNA sequence represented by SEQ ID NO: 39. In addition, the nucleotide sequence encoding the amino acid sequence represented by SEQ ID NO: 40 may be the DNA sequence represented by SEQ ID NO: 41, and the nucleotide sequence encoding the amino acid sequence represented by SEQ ID NO: 42 may be the DNA represented by SEQ ID NO: 43.

In addition, the gene encoding IL-10 may be a nucleotide sequence encoding an IL-10 variant that retains the activity of IL-10. The nucleotide sequence encoding the IL-10 variant may be a nucleotide sequence encoding an amino acid sequence having a sequence homology of at least 60% or more, 70% or more, 80% or more, 90% or more to the IL-10 amino acid sequence shown above, and most preferably, may be a nucleotide sequence encoding an amino acid sequence having a sequence homology of 95% or more.

The nucleotide sequence encoding the IL-10 variant may be a nucleotide sequence having a sequence homology of at least 60% or more, 70% or more, 80% or more, 90% or more to the IL-10 nucleotide sequence shown above, and most preferably, may be a nucleotide sequence having a sequence homology of 95% or more.

The term “GDNF” as used herein refers to a protein constituting the GDNF ligand family. The GDNF ligand family consists of GDNF, neurturin (NRTN), artemin (ARTN), and persephin (PSPN). In addition, GDNF is a protein that promotes the survival of many kinds of neurons and transmits signals through the GFRal receptor. GDNF may be a protein derived from a human, a rat, a dog, a cat, or a horse, but is not limited thereto. The gene encoding GDNF may be the nucleotide sequence encoding the amino acid sequence represented by SEQ ID NO: 11, 44, 46, or 48.

Specifically, the nucleotide sequence encoding the amino acid sequence represented by SEQ ID NO: 11 may be the DNA sequence represented by SEQ ID NO: 12 or 13, and may be the mRNA sequence shown in NCBI Reference Sequence: NM_199231.2. In addition, the nucleotide sequence encoding the amino acid sequence represented by SEQ ID NO: 44 may be the DNA sequence represented by SEQ ID NO: 45. The nucleotide sequence encoding the amino acid sequence represented by SEQ ID NO: 46 may be the DNA sequence represented by SEQ ID NO: 47, and the nucleotide sequence encoding the amino acid sequence represented by SEQ ID NO: 48 may be the DNA sequence represented by SEQ ID NO: 49.

In addition, the gene encoding GDNF may be a nucleotide sequence encoding a GDNF variant which retains the GDNF activity. The nucleotide sequence encoding the GDNF variant may be a nucleotide sequence encoding an amino acid sequence having a sequence homology of at least 60% or more, 70% or more, 80% or more, or 90% or more to the GDNF's amino acid sequence shown above, and most preferably, may be a nucleotide sequence encoding an amino acid sequence having a sequence homology of 95% or more.

In addition, the nucleotide sequence encoding the GDNF variant may be a nucleotide sequence having a sequence homology of at least 60% or more, 70% or more, 80% or more, 90% or more to the GDNF nucleotide sequence shown above, and most preferably, may be a nucleotide sequence having a sequence homology of 95% or more.

GABA, the product of GAD gene, has the effect of blocking pain signal transduction, but excessive amounts can cause symptoms such as itching, dizziness, drowsiness, etc., as well as the side effects such as increase in the heart rate or respiratory rate (Longo, Am Fam Physician, 2000).

IL-10 is known to be a cytokine which shows anti-inflammatory actions, but side effects such as flu symptoms and the like can occur (Friedrich, J Invest Dermatol, 2002).

Furthermore, it is known that the expression of GDNF exhibits analgesic efficacies on a variety of pains such as neuropathic pain and the like, but it has been reported in monkey experiments that administration in excess caused neuronal damage of brain (Hovland, Toxicol Pathol, 2007).

A pharmaceutical composition of the present invention can exhibit analgesic actions with a small amount of genes or carriers containing the same. The composition of the present invention consists of a vector containing a gene encoding GAD, a vector containing a gene encoding an anti-inflammatory cytokine in nervous tissues, and/or a vector containing a gene encoding GDNF. And by co-administering substances having different analgesic mechanisms, it is possible to achieve the same or better pain alleviation or treatment effects at a dosage lower than that of individual administration.

Particularly, according to the present invention, when two or more genes selected from the group consisting of genes encoding GAD65, IL-10, and GDNF are co-administered, a synergistic pain-alleviating effect takes place. Therefore, the pharmaceutical composition of the present invention can be useful for alleviating or treating pain.

According to one embodiment of the present invention, the first vector, the second vector, and/or the third vector may be an adeno-associated virus. The adeno-associated virus is not limited to a particular serotype, and preferably may be any one of AAV1 to AAV9.

The pain may be selected from the group consisting of nociceptive pain, psychogenic pain, inflammatory pain, pathological pain, neuropathic pain, cancer pain, postoperative pain, trigeminal neuralgia pain, idiopathic pain, diabetic neuropathic pain, or migraine. In a specific example, the pain may be lumbosacral radiculopathy (LSR).

The inflammatory pain refers to the pain associated with a tissue damage and infiltration of immune cells. In addition, the pathological pain means a disease state in which pain is caused by damage to a nerve tissue or its abnormal function. Also, the pathological pain may be dysfunctional pain, such as fibromyalgia, irritable bowel syndrome, or tension headache.

In addition, pain can include back pain which can be anatomically distinguished: neck pain, middle back pain, lower back pain, or tailbone pain. In addition, the pain may be at least one selected from the group consisting of neuropathic pain, cancer pain, postoperative pain, trigeminal neuralgia pain, idiopathic pain, diabetic neuropathic pain, migraine, and the like. In a specific example, the pain may be lumbosacral radiculopathy.

Neuropathic pain can be caused by a damage or disease that affects the somatosensory system. Neuropathic pain can be an abnormal sensation called allodynia and dysesthesia. In addition, the general characteristics of neuropathic pain include the sense of hot or cold, pins and needles, numbness, and itching. In contrast, nociceptive pain is often expressed as aching.

In addition, migraine is associated with a number of autonomic nervous system symptoms, and is a chronic disorder that causes headaches of normal to serious severities. Migraine is known to be associated with increased excitability of the cerebral cortex and abnormal regulation of pain neurons in the trigeminal nucleus of the brainstem (Noseda, Pain, 2013).

Specifically, a pharmaceutical composition of the present invention can be used for alleviating or treating neuropathic pain and chronic cancer pain.

As used herein, the term “alleviating or treating” means any action that improves or alters pain symptom in a beneficial way by administering the composition of the present invention.

The pharmaceutical composition of the present invention may further comprise a physiologically acceptable carrier. In addition, the pharmaceutical compositions of the present invention may further comprise suitable excipients and diluents conventionally used in the preparation of pharmaceutical compositions. In addition, the compositions of the present invention may be used by preparing them as oral formulations such as powders, granules, tablets, capsules, suspensions, emulsions, syrups, aerosols, etc., external formulations, suppositories, or injections by general methods. Specifically, the pharmaceutical composition may be in the form of an injection. As for the suitable formulations known in the art, those listed in Remington's Pharmaceutical Science (1985) may be used.

In addition, the pharmaceutical composition may comprise a salt (sodium chloride), lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, mineral oil, etc., as carriers, excipients, and diluents. For the formulation of the pharmaceutical composition of the present invention, generally used diluents or excipients such as fillers, extenders, binders, humectants, disintegrators, surfactants, etc. may be utilized.

Formulations for parenteral administration may include sterile solutions, non-aqueous solvents, suspensions, emulsions, freeze-dried formulations, and suppositories. Propylene glycol, polyethylene glycol, vegetable oil such as olive oil, and injectable ester such as ethylolate, etc., may be used for non-aqueous solvents and suspensions. Witepsol, macrogol, Tween 61, cacao oil, laurin oil, glycerogelatin, etc. may be used for suppository bases.

The present invention also provides a method for alleviating or treating pain, comprising administering a pharmaceutical composition comprising two or more selected from the group consisting of genes encoding GAD, IL-10, and GDNF to a subject in need thereof.

The pain is as described above with regard to the pharmaceutical composition.

The subject may be a mammal including a human, or a cell and/or tissue isolated from a mammal including a human. The term “non-human animal” as used herein is intended to encompass all vertebrate animals, which include mammals and non-mammals such as primates, sheep, dogs, cats, horses, cows, chickens, amphibians, reptiles, etc.

As for the administration route, dosage, and administration frequency, the pharmaceutical composition may be administered to a subject in various ways and amounts depending on the condition of a patient and the presence or absence of side effects, and the range of optimal administration methods, dosages and administration frequencies may be appropriately selected by those having ordinary skill in the art. In addition, the pharmaceutical composition may be administered in combination with another drug or a physiologically active substance which is known to show a therapeutic efficacy on a disorder to be treated. Also, the pharmaceutical composition may be prepared in the form of a combination formulation.

Specifically, the pharmaceutical composition of the present invention may be provided in the form of an injection. For example, subcutaneous injection, intramuscular injection, intravenous injection, epidural injection, or intrathecal injection, and the like may be included. Specifically, the pharmaceutical composition may be administered via epidural injection or intrathecal injection, and more specifically, it may be administered via transforaminal epidural injection or intrathecal injection.

As used herein, the term “transforaminal epidural injection” refers to a method of injecting a drug into the inside of an intervertebral foramen which is a space where nerves emerge from the spinal cord through the space between spinal bones, and into the space outside of the dura which surrounds the spinal cord and spinal nerves. In one embodiment, if the pharmaceutical composition of the present invention is made of viruses, the drug can be administered to the inside of the intervertebral foramen of a subject by conducting epidural injection therapy.

As used herein, the term “intrathecal injection” refers to a method of administration by injecting a drug to a space inside dura in the spinal canal. In one embodiment, if the pharmaceutical composition of the present invention is made of plasmids, the drug can be administered to the inside of the spinal canal of a subject by conducting intrathecal injection therapy.

Specifically, if the pharmaceutical composition is made of viral vectors, it can be administered in an amount of 1.0×10⁶ to 1.0×10¹⁴ vg on an adult basis. In addition, when there are two types of viruses to be administered, each type of the viruses can be administered in an amount of 5.0×10⁵ to 5.0×10¹³ vg. If there are three types of viruses to be administered, each type of the viruses can be administered in an amount of 3.0×10⁵ to 3.0×10¹³ vg.

In addition, if the pharmaceutical composition is made of non-viral vectors, it can be administered in a concentration of 0.1 mg/ml to 10 mg/ml, on an adult basis. Also, if the pharmaceutical composition is made of plasmid vectors, the dosage may be 0.1 ml, 1 ml, 2 ml, 3 ml, 4 ml, 5 ml, 6 ml, 7 ml, 8 ml, 9 ml, 10 ml or more, including all values and ranges between them.

As for the administration frequency of a viral vector, it may be administered once or more, or 1 to 10 times. Also, it may be administered at the interval of 1 day to 1 month, or 1 month to 1 year in the case of repeated administration. In addition, if the pharmaceutical composition is made of non-viral vectors, it may be administered 1 or more, or 1 to 10 times. Also, it may be administered at the interval of 12 to 24 hours or 1 to 14 days in the case of repeatedly administration.

The present invention provides a use of the pharmaceutical composition of the present invention for alleviating or treating pain.

The present invention provides a use of the pharmaceutical composition of the present invention for preparing a therapeutic agent for alleviating or treating pain.

### MODES FOR CARRYING OUT THE INVENTION

Hereinafter, the present invention will be described in detail with reference to examples. However, the following examples are for illustrative purposes only and are not intended to limit the scope of the present invention.

### EXAMPLE 1

### Preparation and Property Analysis of Recombinant Adeno-Associated Virus

Adeno-associated viruses required for the present invention were constructed and produced on the basis of the AAV helper-free system (Agilent).

### EXAMPLE 1.1

### Construction of pAAV-GAD65 Plasmid

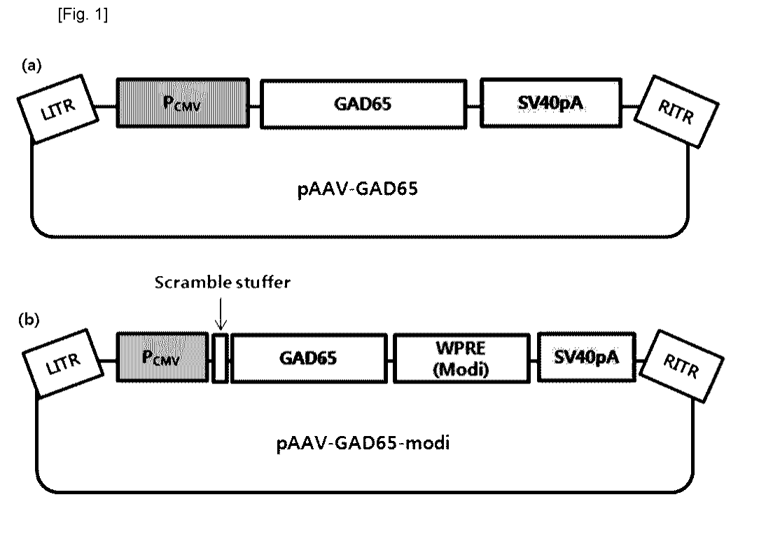

To construct the pAAV-GAD65 plasmid of FIG. 1, the CMV promoter region of pJDK-rGAD65 (Lee, Gene Ther, 2005) was amplified by PCR, and then the resultant was introduced into pGEM-T (Promega) to construct pGEM-T-CMV. The primer sequences used for CMV promoter amplification are as follows:

F-JDK (SEQ ID NO: 15):
5′-TTCGGCCGTCGAGGAGCTTGGCCCATTG-3′
R-JDK (SEQ ID NO: 16):
5′-GACGTCGACCTAGCTAGCGAATTCGGGGCCGCGGAG-3′.

As for the GAD65 gene, the gene represented by SEQ ID NO: 3 was designed by codon-optimization to be suitable for humans based on the human GAD65 (NCBI NM_000818.2) represented by the amino acid sequence of SEQ ID NO: 1, and referred to Bioneer for gene synthesis. The hGAD65 gene introduced into pGEM-T was treated with NheI and SalI to prepare a 1.7 Kb DNA fragment. Thereafter, it was subjected to ligation with the 3.7 Kb DNA fragment obtained by treating pGEM-T-CMV with Nhel and SalI, to complete pGEM-T-CMV-hGAD65 construction.

SV40pA was amplified by conducting PCR using pCI (Invitrogen) as a template, and then the resultant was treated with Clal and SalI to prepare a 222 bp DNA fragment. The DNA fragment was subjected to ligation with the 5.4 Kb DNA fragment prepared by cutting pGEM-T-CMV-hGAD65 with ClaI and SalI, to finally prepare pGEM-T-CMV-hGAD65-SV40pA. The primer sequences used for SV40pA amplification are as follows:

F-SV40pA (SEQ ID NO: 17):
5′-CCATCGATCAGACATGATAAGATACATTGATGAG-3′
R-SV40pA (SEQ ID NO: 18):
5′-GACGTCGACGCGGCCGCTACCACATTTGTAGAGGTTTTACTTG-3′.

To construct an adeno-associated virus vector, the ampicillin resistance gene in pAAV-MCS (Agilent) was replaced with the kanamycin resistance gene. The kanamycin resistance gene was amplified by PCR using pET-28 (a) (Novagen) as a template. The amplified 816 bp kanamycin resistance gene was subjected to ligation with pGEM-T to construct pGEM-T-Kanⁿ. The primer sequences used for kanamycin resistance gene amplification are as follows:

F-Kan (SEQ ID NO: 19):
5′-AGGCGCCATGAGCCATATTCAACGGGAA-3′
R-Kan (SEQ ID NO: 20):
5′-TTCATGATTAGAAAAACTCATCGAGCATC-3′.

To introduce the kanamycin resistance gene, Spel and EcoRV sites were respectively generated by mutagenesis upstream and downstream of the ampicillin resistance gene in pAAV-MCS, and then the resultant was treated with SpeI and EcoRV again. The resultant was subjected to ligation with the DNA fragment obtained by cutting the previously constructed pGEM-T-Kan^{r} with NheI and EcoRV, to construct pAAV-MCS-Kan^{r}.

The constructed pAAV-MCS-Kan^{r} was treated with Notl and BamHI, and then subjected to ligation with the 2.7 Kb DNA fragment obtained by cutting pGEM-T-CMV-hGAD65-SV40pA with EagI and PvuI, to construct pssAAV-GAD65.

To introduce the GAD65 expression cassette into pVAX1 (Invitrogen), the BamHI site was generated by mutagenesis downstream of the bGHpA. Then, the resultant was cut with MluI and NheI to prepare DNA fragments. The LITR and CMV promoter regions were amplified by PCR using pssaAV-GAD65 as a template and cloned into pGEM-T easy (Promega). Thereafter, the resultant was cut with AscI and NheI, and subjected to ligation with the pVAX1 vector previously prepared, to construct pVAX1-LITR-CMV. Primer sequences used for LITR and CMV promoter region amplification are as follows:

F-ITR (SEQ ID NO: 21):
5′-ATGGCGCGCCCCTGGCCTTTTGCTGGCC-3′,
R-JDK (SEQ ID NO: 16):
5′-GACGTCGACCTAGCTAGCGAATTCGGGGCCGCGGAG-3′.

pVAX1-LITR-CMV was cut with NotI and NheI again to prepare DNA fragments. PSSAAV-GAD65 was cut with EagI and NheI, and subjected to ligation with the DNA fragments previously prepared, to construct pVAX1-LITR-CMV-hGAD65-SV40pA.

The pVAX1-LITR-CMV-hGAD65-SV40pA was cut with Hpal and BamHI to prepare DNA fragments. In addition, psA-SV40pA-RITR, which had been prepared by amplifying through PCR using pssaAV-GAD65 as a template and cloning into pGEM-T easy, was treated with Hpal and BamHI, to prepare DNA fragments. The two DNA fragments were ligated to complete pVAX1-LITR-CMV-hGAD65-SV40pA-RITR (hereinafter abbreviated as “pAAV-GAD65”). Primer sequences used for SV40pA and RITR region amplification are as follows:

F-SV40pA (SEQ ID NO: 17):
5′-CCATCGATCAGACATGATAAGATACATTGATGAG-3′
R-ITR (SEQ ID NO: 22):
5′-ATGGATCCGCTAGTAAATACCGCATCAG-3′.

The schematic diagram of the pAAV-GAD65 plasmid is shown in FIG. 1.

The following procedure was carried out to construct modified pAAV-GAD65.

First, the vector was cut with Nhel, and then an arbitrary random nucleotide sequence was inserted between the CMV promoter and GAD65 gene by an infusion method. The inserted nucleotide sequences are as follows:

Scramble stuffer (SEQ ID NO: 29):
5′-GTCGACGGTATCGATAAGCTTGATATCGAATTCCTGCAGCCC-3′
Stuffer_scramble_F (SEQ ID NO: 30):
5′-CTAGGTCGACGGTATCGATAAGCTTGATATCGAATTCCTGCAGCC
C-3′
Stuffer_scramble_R (SEQ ID NO: 31):
5′-CTAGGGGCTGCAGGAATTCGATATCAAGCTTATCGATACCGTCGA
C-3′.

Next, the WPRE nucleotide sequence (Schambach, Gene Ther, 2006), from which the X-protein region which can provide an oncogenic effect was removed, was amplified by PCR, and inserted at the back of GAD65 gene using Pac1 and Hpa1 restriction enzymes. At the same time, some portion of SV40pA was removed to construct a modified SV40pA. The primer sequences used for WPRE amplification are as follows:

WPRE_Pac1_F (SEQ ID NO: 25):
5′-GGTGGTTTAATTAAAATCAACCTCTGGATTACAAAATTTG-3′
WPRE_modi_Hpa1_R (SEQ ID NO: 26):
5′-GGTGGTGTTAACGACAACACCACGGAATTG-3′.

The finally modified plasmid was pVAX1-LITR-CMV-scramble stuffer-hGAD65-WPRE (modi)-SV40pA (modi)-RITR (hereinafter abbreviated as “pAAV-GAD65-modi”), and its schematic diagram is shown in FIG. 1.

### EXAMPLE 1.2

### Construction of pAAV-IL-10 Plasmid

The pAAV-IL-10 plasmid was constructed by the same method as in Example 1.1. As for the rat IL-10 gene, the gene represented by the nucleotide sequence of SEQ ID NO: 8 was designed by codon-optimization to be suitable for rats based on the human IL-10 (NCBI NM-012854) represented by the amino acid sequence of SEQ ID NO: 6, and referred to Bioneer for gene synthesis. The rIL-10 gene was amplified by conducting PCR using the rat IL-10 gene introduced into pGEM-T easy as a template, and then the resultant was treated with NheI and SalI to prepare a 0.5 Kb DNA fragment. In addition, PGEM-T-CMV was treated with NheI and SalI to prepare a 3.7 Kb DNA fragment. The two DNA fragments were ligated to prepare pGEM-T-CMV-rIL-10. The primer sequences used for rIL-10 amplification are as follows:

F-rIL-10 (SEQ ID NO: 23):
5′-CCGCTAGCGCCACCATGCCT-3′
R-rIL-10 (SEQ ID NO: 24):
5′-GACGTCGACGCCATCGATGGCTTAATTAATCAATTCTTC-3′.

As for the SV40pA, the gene was amplified by conducting PCR using pCI as a template and then treated with NotI and SalI to prepare a 222 bp DNA fragment. In addition, the pGEM-T-CMV-rIL-10 was treated with Clal and Sall to prepare a 4.2 Kb DNA fragment. The two DNA fragments were ligated to construct pGEM-T-CMV-rIL-10-SV40pA. The primer sequences used for SV40pA amplification are as follows:

F-SV40pA (SEQ ID NO: 17):
5′-CCATCGATCAGACATGATAAGATACATTGATGAG-3′
R-SV40pA (SEQ ID NO: 18):
5′-GACGTCGACGCGGCCGCTACCACATTTGTAGAGGTTTTACTTG-3′.

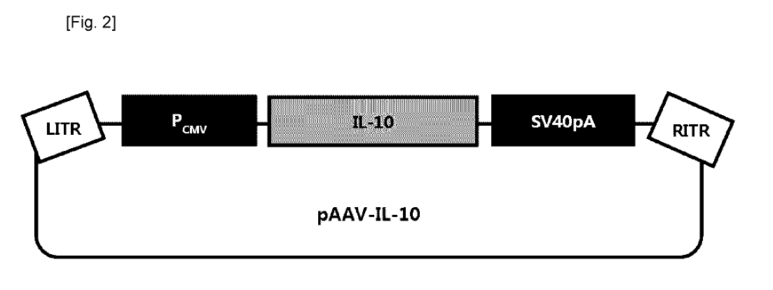

pGEM-T-CMV-rIL-10-SV40pA was treated with EagI to prepare a 1.6 Kb DNA fragment. In addition, pAAV-MCS-Kanⁿ was treated with NotI and BamHI to prepare DNA fragments. Thereafter, the two DNA fragments were ligated to construct pssAAV-CMV-rIL-10-SV40pA (hereinafter abbreviated as “pAAV-IL-10”). The schematic diagram of the pAAV-IL-10 plasmid is shown in FIG. 2.

### Example 1.3.

### Construction of pAAV-GDNF Plasmid

As for the human GDNF gene, the gene represented by the SEQ ID NO: 13 was designed by codon-optimization to be suitable for humans based on human GDNF (NCBI NM_199231.2) represented by the amino acid sequence of SEQ ID NO: 11, and referred to Bioneer for gene synthesis. The hGDNF gene introduced into the pGEM-B1 plasmid was treated with NheI and PacI to prepare a DNA fragment of about 0.6 kb. The pGEM-T-CMV-rIL-10-SV40pA plasmid was treated with NheI and PacI to prepare a 2.8 kb fragment in which the rIL-10 gene was removed. The two DNA fragments were ligated to construct the pGEM-T-CMV-hGDNF-SV40pA plasmid.

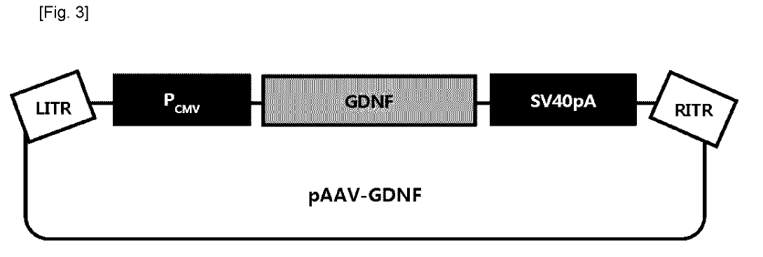

Then, the completed pGEM-T-CMV-hGDNF-SV40pA plasmid was treated with EagI to prepare a 1.5 kb DNA fragment. In addition, pAAV-MCS-Kan^{r} was treated with NotI and BamHI to prepare a 1.8 kb DNA fragment. The two DNA fragments were ligated to construct pssAAV-CMV-hGDNF-SV40pA-Kan^{r} (hereinafter abbreviated as “pAAV-GDNF”). The schematic diagram of the pAAV-GDNF plasmid is shown in FIG. 3.

### EXAMPLE 1.4

### Construction of pAAV-GDNF-IL-10 Plasmid

The CAG promoter (cytomegalovirus enhancer, chicken β-actin promoter, and rabbit β-globin poly A signal) was subjected to PCR amplification using pAxCAwtit2 contained in the Adenovirus dual expression kit (Takara), and treated with ApaI and XbaI to improve expression, thereby removing about 80% of the chicken ß-actin region in the CAG promoter to produce a short CAG (sCAG) promoter (Fagoe, Gene Ther, 2014). As for the human IL-10 gene, the gene encoding the SEQ ID NO: 14 was designed by codon-optimization of the gene encoding SEQ ID NO: 9 to be suitable for humans, and referred to Bioneer for gene synthesis. Next, DNA fragment for bovine growth hormone (bGH) poly A was obtained by PCR amplification. The pVAX1/sCAG-hIL-10-bGHpA was constructed using pVAX1 (Invitrogen) to contain the promoter and poly A and human IL-10 genes.

Next, pVAX1/CMV-hGDNF-SV40pA was prepared by the same method as in the Example 1.1., using the human GDNF gene of the Example 1.3. Thereafter, the SV40pA-hGDNF-CMV gene cassette was amplified by conducting PCR using pVAX1/CMV-hGDNF-SV40pA as a template, and a 1.5 kb DNA fragment was prepared. The primer sequences used for the gene cassette amplification are as follows:

SV40-CMV-sCAG-bGHpA-Infu-F (SEQ ID NO: 27):
5′-CCTGCGGCCGGTCGACTACCACATTTGTAGAGGTTTTACTTGC-3′
SV40-CMV-sCAG-bGHpA-Infu-R (SEQ ID NO: 28):
5′-AATAATCAATGTCGACTCGAGGAGCTTGGCCCATT-3′

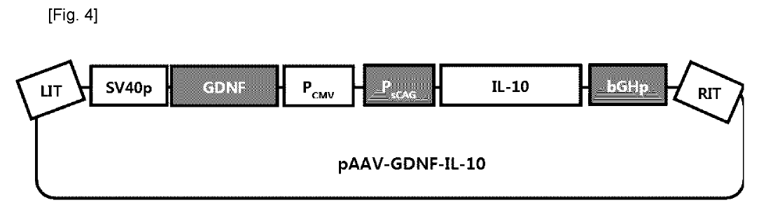

Next, pVAX1/sCAG-hIL-10-bGHpA was treated with Sall to prepare a DNA fragment of about 3.9 kb, and the 1.5 kb DNA fragment described above was inserted into the 3.9 kb DNA fragment using an In-Fusion HD Cloning Kit (Clontech) to construct pVAX 1/SV40pA-hGDNF-CMV-sCAG-hIL-10-bGHpA (hereinafter abbreviated as “pAAV-GDNF-IL-10”). The pAAV-GDNF-IL-10 plasmid is schematically shown in FIG. 4.

### EXPERIMENTAL EXAMPLE 1

### Confirmation of Expression of pAAV-GAD65, pAAV-IL-10, pAAV-GDNF and pAAV-GDNF-IL-10 Plasmids

The pAAV-GAD65, pAAV-IL-10, pAAV-GDNF, or pAAV-GDNF-IL-10 plasmids prepared in the Examples 1.1. to 1.4 were respectively transfected into human embryonic kidney cell line 293T cells using jetPRIME (Polyplus). The transfected cells were cultured in a 37° C. incubator for 48 hours. Thereafter, the cell culture medium or cultured cells were harvested. The cells were dissolved with a solvent, and the prepared samples were treated with each of the antibodies to GAD65 (Merck Millipore), IL-10 (Santa Cruz), and GDNF (R&D systems), and subjected to Western blotting.

Specifically, in the case of pAAV-GAD65, the human embryonic kidney cell line 293T cells were treated with 2μg of pAAV-GAD65 plasmid and cultured for 48 hours. Thereafter, the cultured cells were dissolved and the expression of GAD65 in the cells was confirmed through Western blotting.

In the case of pAAV-IL-10 and pAAV-GDNF plasmids, the human embryonic kidney cell line 293T cells were treated with 1μg of pAAV-IL-10 or pAAV-GDNF plasmid, and cultured for 48 hours. Thereafter, the culture medium was harvested and the expression of IL-10 or GDNF in the medium was confirmed through Western blotting.

In the case of the pAAV-GDNF-IL-10 plasmid, the human embryonic kidney cell line 293T cells were treated with 1μg of pAAV-GDNF-IL-10 plasmid and cultured for 48 hours. Thereafter, the cultured cells were dissolved, and the expression of intracellular IL-10 and GDNF was confirmed through Western blotting.

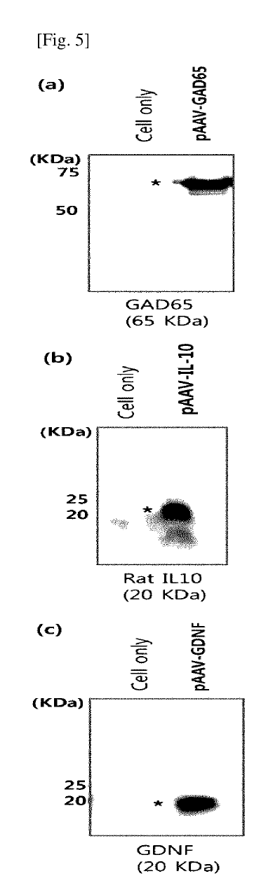

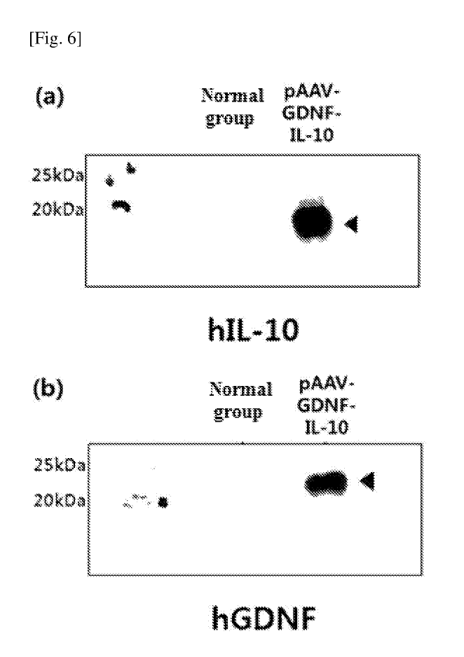

As a result, it was confirmed that the transfected pAAV-GAD65, pAAV-IL-10, pAAV-GDNF or pAAV-GDNF-IL-10 plasmid was expressed (FIGS. 5 and 6).

### Example 2

### Preparation of Recombinant Adeno-Associated Virus

The AAV-IL-10 virus used in the experiment was produced and purified by UNC vector core. The production method is as follows. The pVax-rIL-10, pHelper and pRC5 were transfected into human embryonic kidney cell line 293T cells. Thereafter, the resultant was subject to purification by column chromatography to secure AAVS-IL-10 virus. The titer of the produced virus was measured using qPCR.

The AAV-GAD65 and AAV-GDNF viruses were produced and purified by KRcrogen. The production method is as follows. The AAV-transgenes (pAAV-GAD65 and pAAV-GDNF plasmids) were respectively transfected into the human embryonic kidney cell line 293T cells using the calcium phosphate method along with pHelper and pRC. In the case of GAD65, pRC5 introduced with the capsid gene of serotype 5 was used. In the case of GDNF, pRC1 introduced with the capsid gene of AAV serotype 1 was used. The transfected cells were cultured in a 37° C. incubator and the cells were harvested after 48 hours.

Thereafter, only the bands containing viruses were isolated and purified through the ultrahigh speed centrifugation method according to the cesium concentration gradient, to secure AAV5-GAD65, and AAV1-GDNF viruses. The titers of the produced viruses were measured using qPCR.

The AAV-GDNF-IL-10 virus was produced and purified by Cdmogen. The production method is as follows. The AAV-transgene (pAAV-GDNF-IL-10 plasmid) was transfected into the human embryonic kidney cell line 293T cells using the calcium phosphate method along with pHelper and pRC5. The transfected cells were cultured in a 37° C. incubator and the cells were harvested after 48 hours.

Thereafter, only the bands containing viruses were isolated and purified through ultrahigh speed centrifugation according to the cesium concentration gradient, to secure AAV5-GDNF-IL-10 virus. The titer of the produced virus was measured using qPCR.

The AAV-GAD65-modi virus was produced and purified by Cdmogen. The production method is as follows. The AAV-transgene (pAAV-hGAD65-modi plasmid) was transfected into human embryonic kidney cell line 293T cells using the calcium phosphate method along with pHelper and pRC5. The transfected cells were cultured in a 37° C. incubator and the cells were harvested after 48 hours.

Thereafter, only the bands containing viruses were isolated and purified by ultra-high-speed centrifugation according to the cesium concentration gradient to secure the AAV5-GAD65-modi virus. The titer of the produced virus was measured using qPCR.

### EXPERIMENTAL EXAMPLE 2

### Property Analysis of Recombinant Adeno-Associated Virus

In order to examine the protein expression of recombinant adeno-associated virus delivered into a cell, human embryonic kidney cell line 293T or HeLa cells were treated with the AAV-GAD65, AAV-GAD65-modi, AAV-IL-10 or AAV-GDNF virus obtained above, and protein expression was examined by Western blotting. Specifically, 293T or HeLa cells were seeded at 5×10⁵ cells/well in a 6-well plate. And on the next day, the cells were respectively treated with 3 types of viruses at 10,000 vg/well, and then cultured in a 37° C. incubator. After 48 hours, the cells were harvested and were dissolved with a solvent and the culture medium was concentrated using the amicon (Merck Millipore). Then, the prepared samples were respectively treated with the antibodies to GAD65 (Cell signaling), IL-10 (Santa Cruz) and GDNF (R&D systems), and subjected to Western blotting.

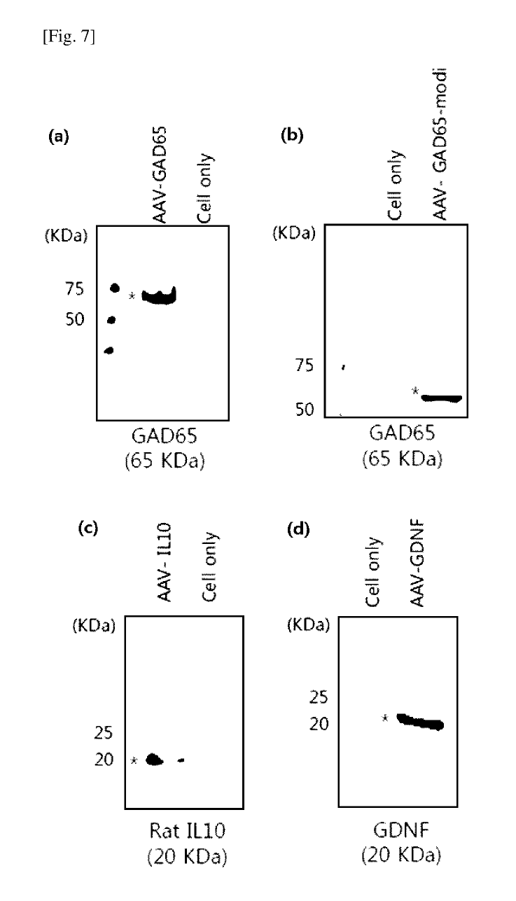

As a result, it was confirmed that each target protein was expressed in the cell lysate of human embryonic kidney cell line 293T or HeLa cell line treated with AAV-GAD65, AAV-GAD65-modi, AAV-IL-10 or AAV-GDNF virus (FIG. 7). Therefore, it was confirmed that there was no abnormality in the structures and properties of the recombinant adeno-associated viruses used in the experiment.

Also, in order to confirm that GABA is produced by AAV-GAD65 or AAV-GAD65-modi virus, the culture medium of the cells treated with the AAV-GAD65 or AAV-GAD65-modi virus was harvested and subjected to GABA ELISA (LDN) analysis. For each experimental group, two identical samples were prepared separately to conduct the analysis, and the bar graph shows the value for each sample.

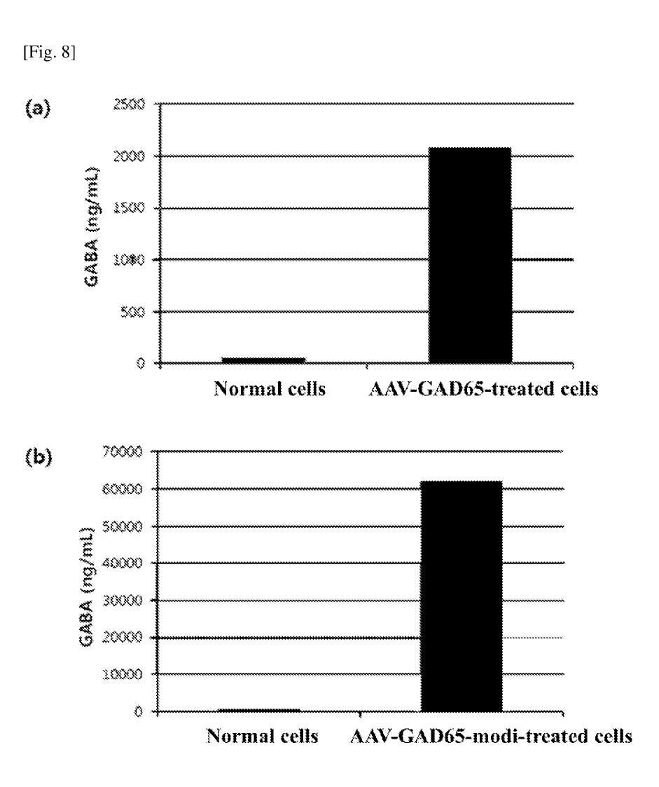

As a result, it was confirmed that GABA was secreted to the culture medium by GAD65 introduced into cells by AAV-GAD65 or AAV-GAD65-modi virus (FIG. 8).

### EXPERIMENTAL EXAMPLE 3

### Comparison of Analgesic Efficacies Between Individual Administration of AAV-GAD65, AAV-IL-10 or AAV-GDNF Virus and Co-Administration of AAV-GAD65 and AAV-GDNF, or AAV-IL-10 and AAV-GDNF Viruses

### EXPERIMENTAL EXAMPLE 3.1

### Preparation of Administration Sample

The viruses prepared in Example 2 were used for the test. 30 minutes prior to the animal administration experiment, the reagents stored at −80° C. were thawed at room temperature and prepared by mixing by a vortexer. AAV-GAD65, AAV-IL-10, AAV-GDNF, and AAV-GFP viruses were diluted in PBS to obtain the titers shown in Table 1. The AAV-GFP virus was administered in the same amount as other recombinant adeno-associated viruses. The GFP is a protein having no analgesic efficacy. The viruses required were mixed according to the contents indicated in Table 1, and administered in an amount of 9.0×10⁸ vg/5 μl per animal (vg: virus genome).

TABLE 1

| | Virus types and contents | | | |
|---|---|---|---|---|
| | AAV- | AAV- | AAV- | AAV- |
| Samples | GAD65 | IL-10 | GDNF | GFP |
| AAV-GFP | — | — | — | 9.0 × 10⁸ |
| | | | | vg/5 μl |
| AAV-GAD65 | 9.0 × 10⁸ | — | — | — |
| | vg/5 μl | | | |
| AAV-IL-10 | — | 9.0 × 10⁸ | — | — |
| | | vg/5 μl | | |
| AAV-GDNF | — | — | 9.0 × 10⁸ | — |
| | | | vg/5 μl | |
| AAV-GAD65 + | 4.5 × 10⁸ | — | 4.5 × 10⁸ | — |
| AAV-GDNF | vg/5 μl | | vg/5 μl | |
| AAV-IL-10 + | — | 4.5 × 10⁸ | 4.5 × 10⁸ | — |
| AAV-GDNF | | vg/2.5 μl | vg/2.5 μl | |

### EXPERIMENTAL EXAMPLE 3.2

### Construction of Neuropathic Pain-Induced Rats and Administration of Samples

150 to 200 g male SD-rats were subjected to inhalation anesthesia. And then the upper part of the calf was incised and both ends of the common peroneal nerve and tibial nerve were tied and knots were made at the interval of 0.5 to 1 cm by 7-0 suture. The regions between the knots of the two nerve bundles were cut with a scissor and the incision site was sutured. Thereafter, the rats were recovered to be awakened from the anesthesia and returned to the cage. Two weeks later, the von Frey filament test was conducted to examine pain induction, and then the samples prepared in Example 2.1 were respectively administered (Decosterd, Pain, 2000).

The samples were administered by the transforaminal epidural injection method at a location adjacent to the dorsal root ganglion (DRG). The pain-induced rat was subjected to inhalation anesthesia, and the vertebrae were exposed by linearly incising the back of the rat at the levels of lumbar spines L3 to L5. At the side of the exposed space, the L4 transverse process, one of the spinal projections, was made visible. The rat was laid down sideways such that its lateral side is visible from above and the L4 intervertebral foramen was visible.

Thereafter, a needle attached to the catheter was inserted into the prepared sample, and a Hamilton syringe was connected to the opposite end of the catheter and pulled to the marking line of 5 μl to inject the sample into the catheter. The Hamilton syringe was removed from the catheter and then the catheter was secured by holding the point 1 cm away from the tip of the needle using Halsted-Mosquito. Then, while holding and pulling the L4 spine upward with tweezers, the tip of the needle secured by Halstead Mosquito was taken around the L4 intervertebral foramen with the other hand. The tip of the needle was inserted into the bent region inside the intervertebral foramen whose space was secured. Then, the needle which was being held was released.

After confirming that the needle was fixed, a 1 ml syringe was connected to the catheter connected to the opposite side of the needle. By gently pressing the piston of the syringe, the sample was slowly injected around the dorsal root ganglion of the rat. Thereafter, the incision site was sutured. 4 weeks after the sample administration, pain responses were observed using von Frey filament test.

### EXPERIMENTAL EXAMPLE 3.3

### Pain Observation Using Von Frey Filament Test

Pain was observed using the von Frey filament test. The method is to calculate the threshold value according to a predetermined pattern of the pain response with a total of eight filaments of 0.4, 0.6, 1, 2, 4, 6, 8 and 15 g.

Pain generating regions were searched by changing the position from the beginning portion of the outermost toe to the heel of the sole where pain was generated. Since the rats suddenly took off the soles and shrank or licked their soles with their mouths when pain was generated, the pain generating regions could be found. If there were reactions three times or more when the surrounding area was pricked five times with the filament of each step, it was regarded as a pain response. And the test was proceeded by replacing the filament with that of the next step. In this way, the pattern of each step was recorded.

The pain patterns were recorded based on the pattern table established by S.R. Chaplan, and the threshold values were calculated using the pain patterns (Chaplan, J Neurosci Methods, 1994). As for the behavioral analysis, the animal groups were blinded at a specified time and at least 3 researchers observed, and the results of recorded patterns were statistically processed, to analyze the pain results.

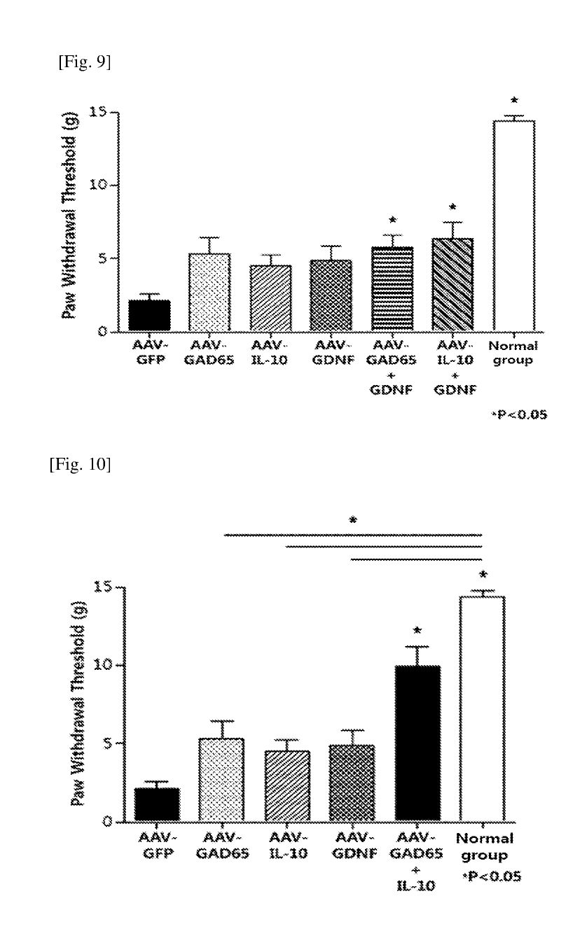

As a result, it was found that the pain-alleviating effect was higher when AAV-GAD65 and AAV-GDNF, or AAV-IL-10 and AAV-GDNF viruses were combined and co-administered as compared to individual administration of AAV-GAD65, AAV-IL-10 or AAV-GDNF virus (FIG. 9).

### EXPERIMENTAL EXAMPLE 4

### Comparison of Analgesic Efficacies Between Individual Administration of AAV-GAD65, AAV-IL-10 or AAV-GDNF Virus and Co-Administration of AAV-GAD65, AAV-IL-10, and AAV-GDNF Viruses

The viruses prepared in Example 2 were used for the test. 30 minutes prior to the animal administration experiment, the reagents stored at −80° C. were thawed at room temperature and prepared by mixing by a vortexer. AAV-GAD65, AAV-IL-10, AAV-GDNF, and AAV-GFP viruses were diluted in PBS to obtain the titers shown in Table 2. The AAV-GFP virus was administered in the same amount as other recombinant adeno-associated viruses. The GFP is a protein whose analgesic efficacy has not been reported. The viruses were mixed according to the contents shown in Table 2, and administered in an amount of 9.0×10⁸ vg/5 μl per animal.

TABLE 2

| | Virus types and contents | | | |
|---|---|---|---|---|
| | AAV- | AAV- | AAV- | AAV- |
| Samples | GAD65 | IL-10 | GDNF | GFP |
| AAV-GFP | — | — | — | 9.0 × 10⁸ |
| | | | | vg/5 μl |
| AAV-GAD65 | 9.0 × 10⁸ | — | — | — |
| | vg/5 μl | | | |
| AAV-IL-10 | — | 9.0 × 10⁸ | — | — |
| | | vg/5 μl | | |
| AAV-GDNF | — | — | 9.0 × 10⁸ | — |
| | | | vg/5 μl | |
| AAV-GAD65 + | 3.0 × 10⁸ | 3.0 × 10⁸ | 3.0 × 10⁸ | — |
| AAV-IL-10 + | vg/5 μl | vg/5 μl | vg/5 μl | |
| AAV-GDNF | | | | |

To the pain animal model produced by the same method as in Experimental Example 3.2., samples prepared according to the virus contents shown in Table 2 were administered. Thereafter, the von Frey filament test was conducted by the same method as in Experimental Example 3.3. to observe the pain responses.

As a result, it was found that the pain-alleviating effect was higher when all of the AAV-GAD65, AAV-IL-10 and AAV-GDNF viruses were co-administered as compared to individual administration of AAV-GAD65, AAV-IL-10 or AAV-GDNF virus (FIG. 10).

### EXPERIMENTAL EXAMPLE 5

### Comparison of Analgesic Efficacies Between Co-Administration of AAV-GAD65 and AAV-GDNF, or AAV-IL-10 and AAV-GDNF Viruses and Co-Administration of AAV-GAD65, AAV-IL-10, and AAV-GDNF Viruses

The adeno-associated viruses prepared in Example 2 were used for the test. 30 minutes prior to the animal administration experiment, the reagents stored at −80° C. were thawed at room temperature and prepared by mixing by a vortexer. AAV-GAD65, AAV-IL-10, AAV-GDNF, and AAV-GFP viruses were diluted in PBS to obtain the titers shown in Table 3. The AAV-GFP was administered in the same amount as other recombinant adeno-associated viruses. The GFP is a protein which has no analgesic efficacy. The viruses required were mixed according to the contents shown in Table 3, and administered in an amount of 9.0×10⁸ vg/5 μl per animal.

TABLE 3

| | Virus types and contents | | | |
|---|---|---|---|---|
| | AAV- | AAV- | AAV- | AAV- |
| Samples | GAD65 | IL-10 | GDNF | GFP |
| AAV-GFP | — | — | — | 9.0 × 10⁸ |
| | | | | vg/5 μl |
| AAV-GAD65 + | 4.5 × 10⁸ | — | 4.5 × 10⁸ | — |
| AAV-GDNF | vg/5 μl | | vg/5 μl | |
| AAV-IL-10 + | — | 4.5 × 10⁸ | 4.5 × 10⁸ | — |
| AAV-GDNF | | vg/5 μl | vg/5 μl | |
| AAV-GAD65 + | 3.0 × 10⁸ | 3.0 × 10⁸ | 3.0 × 10⁸ | — |
| AAV-IL-10 + | vg/5 μl | vg/5 μl | vg/5 μl | |
| AAV-GDNF | | | | |

To the pain animal model produced by the same method as in Experimental Example 3.2., samples prepared according to the virus contents shown in Table 3 were administered. Thereafter, the von Frey filament test was conducted by the same method as in Experimental Example 3.3. to observe the pain responses.

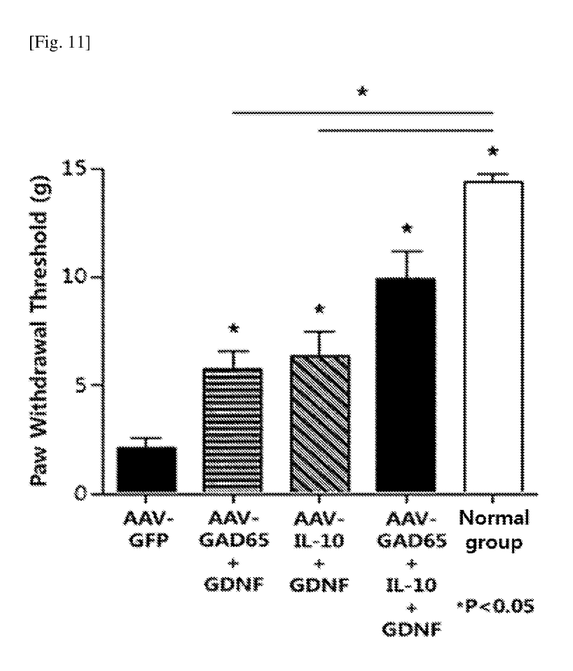

As a result, it was found that the pain-alleviating effect was higher when all of the AAV-GAD65, AAV-IL-10 and AAV-GDNF viruses were co-administered as compared to co-administration of AAV-GAD65 and AAV-GDNF, or AAV-IL-10 and AAV-GDNF viruses (FIG. 11).

### EXPERIMENTAL EXAMPLE 6

### Comparison of Analgesic Efficacies Between Individual Administration of pAAV-GAD65, pAAV-IL-10, or pAAV-GDNF Plasmid and Co-Administration of pAAV-GAD65 and pAAV-GDNF, or pAAV-IL-10 and pAAV-GDNF Plasmids

### EXPERIMENTAL EXAMPLE 6.1

### Preparation of Administration Samples

The plasmids prepared in Example 1 were used for the test. 30 minutes prior to the animal administration experiment, the reagents stored at −80° C. were thawed at room temperature and prepared by mixing by a vortexer. The pAAV-GAD65, pAAV-IL-10, pAAV-GDNF, and pVAX1 plasmids were diluted in the Tris-EDTA buffer to obtain the concentrations shown in Table 4. The pVAX1 plasmid was administered in the same amount as the other plasmids. The pVAX1 plasmid has not been reported to have an analgesic efficacy. The required plasmids were mixed according to the contents shown in Table 4, and administered in an amount of 30 μg/50 μl per animal.

TABLE 4

| | Plasma types and contents | | | |
|---|---|---|---|---|
| | pAAV- | pAAV- | pAAV- | |
| Samples | GAD65 | IL-10 | GDNF | pVAX1 |
| pVAX1 | — | — | — | 30 μg/50 μl |
| pAAV-GAD65 | 30 μg/50 μl | — | — | — |
| pAAV-IL-10 | — | 30 μg/50 μl | — | — |
| pAAV-GDNF | — | — | 30 μg/50 μl | — |
| pAAV- | 15 μg/50 μl | — | 15 μg/50 μl | — |
| GAD65 + | | | | |
| pAAV-GDNF | | | | |
| pAAV-IL-10 + | — | 15 μg/50 μl | 15 μg/50 μl | — |
| pAAV-GDNF | | | | |

### EXPERIMENTAL EXAMPLE 6.2

### Construction of Neuropathic Pain Animal Model and Sample Administration

150 to 200 g male SD-rats were subjected to inhalation anesthesia. And then the upper part of the calf was incised and both ends of the common peroneal nerve and tibial nerve were tied and knots were made at the interval of 0.5 to 1 cm by 7-0 suture. The regions between the knots of the two nerve bundles were cut with a scissor and the incision site was sutured. Thereafter, the rats were recovered to be awakened from the anesthesia and returned to the cage. Two weeks later, the von Frey filament test was conducted to examine pain induction, and then the samples prepared in Example 6.1 were administered (Decosterd, Pain, 2000).

The samples were administered by the intrathecal injection method. The pain-induced rat was subjected to inhalation anesthesia, and the spinous process was exposed by linearly incising the back of the rat at the region of lumbar spines L5. A 50 ml tube was placed under the rat to widen the space between L5 and L6, and a needle of 27 G×13 mm size was inserted. Thereafter, a 1 mL syringe filled with 50 μl of the sample was connected to the needle. The sample was slowly injected by slightly pressing the piston of the syringe. Thereafter, the incision was sutured and this step was finished. One day after the substance administration, the pain responses were observed using the von Frey filament test.

### EXPERIMENTAL EXAMPLE 6.3

### Pain Observation Using Von Frey Filament Test

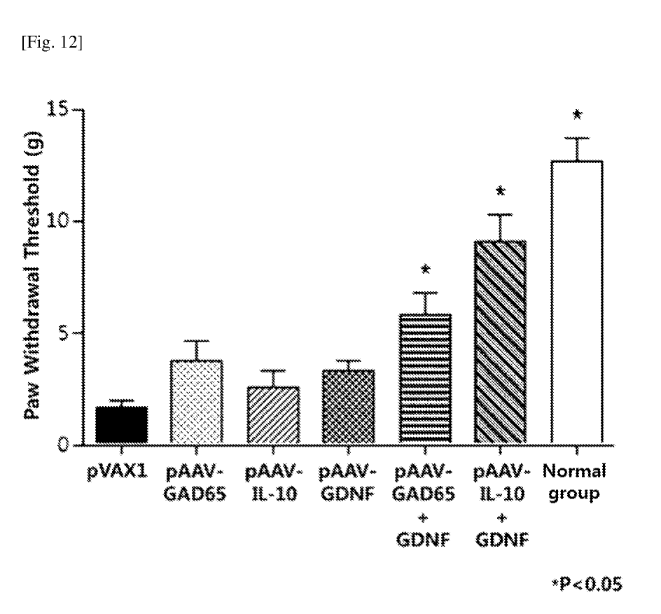

The von Frey filament test was carried out by the same method as in Experimental Example 3.3. As a result, it was found that the pain-alleviating effect was higher when pAAV-GAD65 and pAAV-GDNF, or pAAV-IL-10 and pAAV-GDNF plasmids were co-administered as compared to individual administration of pAAV-GAD65, pAAV-IL-10 or pAAV-GDNF plasmid (FIG. 12).

### EXPERIMENTAL EXAMPLE 7

### Comparison of Analgesic Efficacies Between Individual Administration of pAAV-GAD65, pAAV-IL-10, or pAAV-GDNF Plasmid and Co-Administration of pAAV-GAD65, pAAV-IL-10 and pAAV-GDNF Plasmids

The plasmids prepared in Example 1 were used for the test. 30 minutes prior to the animal administration experiment, the reagents stored at −80° C. were thawed at room temperature and prepared by mixing by a vortexer. The pAAV-GAD65, pAAV-IL-10, pAAV-GDNF, and pVAX1 plasmids were diluted in the Tris-EDTA buffer to obtain the concentrations shown in Table 5. The pVAX1 plasmid was administered in the same amount as the other plasmids. The pVAX1 plasmid has not been reported to have an analgesic efficacy. The required plasmids were mixed according to the contents shown in Table 5, and administered in an amount of 30 μg/50 μl per animal.

TABLE 5

| | Plasma types and contents | | | |
|---|---|---|---|---|
| | pAAV- | pAAV- | pAAV- | |
| Samples | GAD65 | IL-10 | GDNF | pVAX1 |
| pAAV-VAX1 | — | — | — | 30 μg/50 μl |
| pAAV-GAD65 | 30 μg/50 μl | — | — | — |
| pAAV-IL-10 | — | 30 μg/50 μl | — | — |
| pAAV-GDNF | — | — | 30 μg/50 μl | — |
| pAAV- | 10 μg/50 μl | 10 μg/50 μl | 10 μg/50 μl | — |
| GAD65 + | | | | |
| pAAV-IL-10 + | | | | |
| pAAV-GDNF | | | | |

To the pain animal model produced by the same method as in Experimental Example 6.2., samples prepared according to the virus contents shown in Table 5 were administered. Thereafter, the von Frey filament test was conducted by the same method as in Experimental Example 3.3. to observe the pain responses.

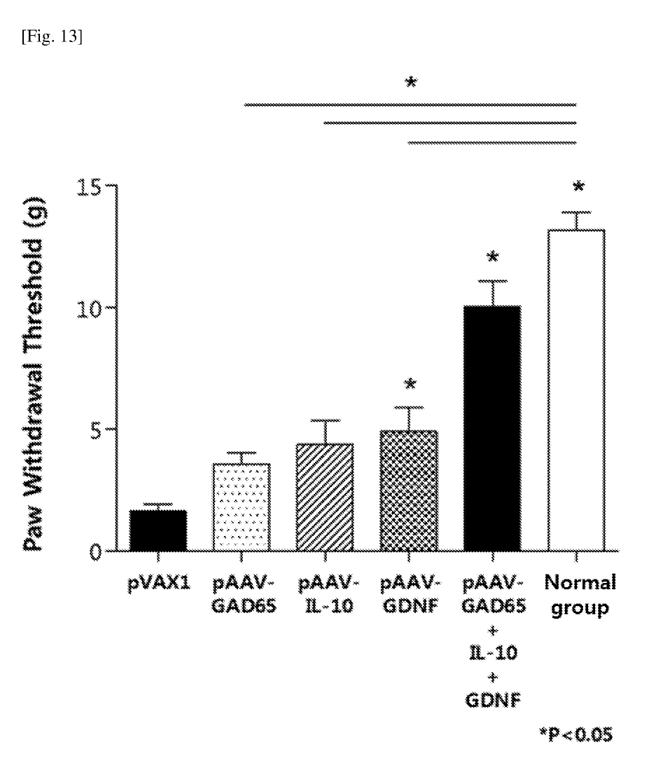

As a result, it was found that the pain-alleviating effect was higher when all of the pAAV-GAD65, pAAV5-IL-10, and pAAV5-GDNF plasmids were co-administered as compared to individual administration of pAAV-GAD65, pAAV-IL-10 or pAAV-GDNF plasmid (FIG. 13).

### EXPERIMENTAL EXAMPLE 8

### Comparison of the Analgesic Efficacies Between Co-Administration of pAAV-GAD65 and pAAV-GDNF, or pAAV-IL-10 and pAAV-GDNF Plasmids and Co-Administration of pAAV-GAD65, pAAV-IL-10 and pAAV-GDNF Plasmids

The plasmids prepared in Example 1 were used for the test. 30 minutes prior to the animal administration experiment, the reagents stored at −80° C. were thawed at room temperature and prepared by mixing by a vortexer. The pAAV-GAD65, pAAV-IL-10, pAAV-GDNF, and pVAX1 plasmids were diluted in the Tris-EDTA buffer to obtain the concentrations shown in Table 6. The pVAX1 plasmid was administered in the same concentration and amount as the other plasmids. The pVAX1 plasmid has not been reported to have an analgesic efficacy. The required plasmids were mixed according to the contents shown in Table 6, and administered in an amount of 30 μg/50 μl per animal.

TABLE 6

| | Plasma types and contents | | | |
|---|---|---|---|---|
| | pAAV- | pAAV- | pAAV- | |
| Samples | GAD65 | IL-10 | GDNF | pVAX1 |
| pVAX1 | — | — | — | 30 μg/50 μl |
| pAAV- | 15 μg/50 μl | — | 15 μg/50 μl | — |
| GAD65 + | | | | |
| pAAV-GDNF | | | | |
| pAAV-IL-10 + | — | 15 μg/50 μl | 15 μg/50 μl | — |
| pAAV-GDNF | | | | |
| pAAV- | 10 μg/50 μl | 10 μg/50 μl | 10 μg/50 μl | — |
| GAD65 + | | | | |
| pAAV-IL-10 + | | | | |
| pAAV-GDNF | | | | |

To the pain animal model produced by the same method as in Experimental Example 6.2., samples prepared according to the plasma contents shown in Table 6 were administered. Thereafter, the von Frey filament test was conducted by the same method as in Experimental Example 6.3. to observe the pain responses.

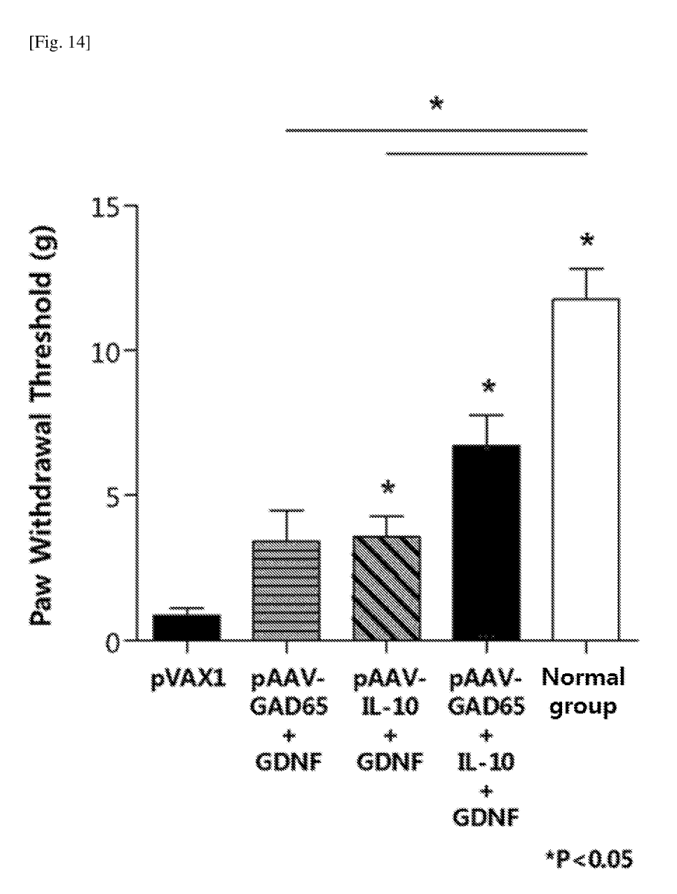

As a result, it was found that the pain-alleviating effect was higher when all of the pAAV-GAD65, pAAV-IL-10 and pAAV-GDNF plasmids were co-administered as compared to co-administration of pAAV-GAD65 and pAAV-GDNF or pAAV-IL-10 and pAAV-GDNF plasmids (FIG. 14).

### EXPERIMENTAL EXAMPLE 9

### Comparison of Analgesic Efficacies of Co-Administration of AAV-GAD65-modi and AAV-GDNF-IL-10 Viruses and Co-Administration of AAV-GAD65, AAV-IL-10 and AAV-GDNF Viruses

The adeno-associated viruses prepared in Example 2 were used for the test. 30 minutes prior to the animal administration experiment, the reagents stored at −80° C. were thawed at room temperature and prepared by mixing by a vortexer. AAV-GAD65-modi, AAV-GDNF-IL-10, AAV-GAD65, AAV-IL-10 and AAV-GDNF viruses were diluted in PBS to obtain the titers shown in Table 7. The viruses required were mixed according to the contents shown in Table 7, and administered in an amount of 1.0×10⁹ vg/5 μlo or 1.5×10⁹ vg/5 μl per animal.

TABLE 7

| | Virus types and contents | | | | |
|---|---|---|---|---|---|
| | AAV- | AAV-GDNF- | AAV- | AAV- | AAV- |
| Samples | GAD65-modi | IL-10 | GAD65 | IL-10 | GDNF |
| Control | — | — | — | — | — |
| AAV-GAD65- | 5.0 × 10⁸ | 5.0 × 10⁸ | — | — | — |
| modi + | vg/5 μl | vg/5 μl | | | |
| AAV-GDNF- | | | | | |
| IL-10 | | | | | |
| AAV-GAD65 + | — | — | 5.0 × 10⁸ | 5.0 × 10⁸ | 5.0 × 10⁸ |
| AAV-IL-10 + | | | vg/5 μl | vg/5 μl | vg/5 μl |
| AAV-GDNF | | | | | |

To the pain animal model produced by the same method as in Experimental Example 3.2., samples prepared according to the virus contents shown in Table 7 were administered. Thereafter, the von Frey filament test was conducted by the same method as in Experimental Example 3.3. to observe the pain responses.

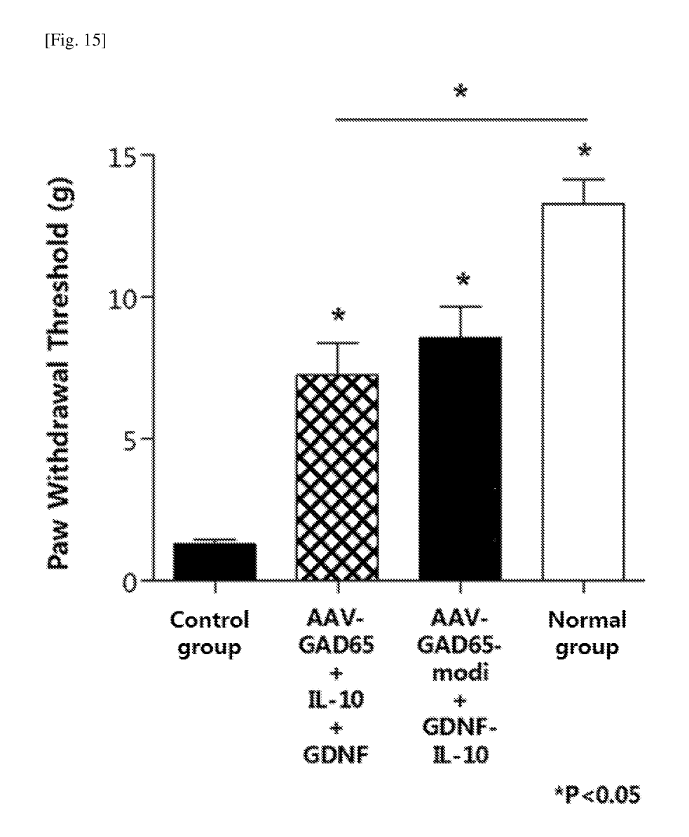

As a result, it was found that the pain-alleviating effect was higher when AAV-GAD65-modi and AAV-GDNF-IL-10 viruses were co-administered as compared to co-administration of all of AAV-GAD65, AAV-IL-10 and AAV-GDNF viruses (FIG. 15).

1. A pharmaceutical composition comprising two or more selected from the group consisting of a gene encoding glutamate decarboxylase (GAD), a gene encoding interleukin-10 (IL-10), and a gene encoding a glial cell-derived neurotrophic factor (GDNF).

2. The pharmaceutical composition of claim 1, wherein the gene is in a form of being operably contained in a vector.

3. The pharmaceutical composition of claim 2, wherein the vector is at least one viral vector selected from the group consisting of adenovirus, adeno-associated virus, herpes simplex virus, lentivirus, retrovirus, and poxvirus.

4. The pharmaceutical composition of claim 2, wherein the vector is at least one non-viral vector selected from the group consisting of a plasmid, a lipo some, a cationic polymer, a micelle, an emulsion, and solid lipid nanoparticles.

5. The pharmaceutical composition of claim 1, wherein the GAD is GAD65 or GAD67.

6. The pharmaceutical composition of claim 1, wherein the gene encoding GAD is a nucleotide sequence encoding the amino acid sequence of SEQ ID NO: 1, SEQ ID NO: 4, SEQ ID NO: 32, SEQ ID NO: 34 or SEQ ID NO: 36.

7. The pharmaceutical composition of claim 1, wherein the gene encoding GAD is the nucleotide sequence of SEQ ID NO: 2, SEQ ID NO: 3, SEQ ID NO: 5, SEQ ID NO: 33, SEQ ID NO: 35, SEQ ID NO: 37.

8. The pharmaceutical composition of claim 1, wherein the gene encoding IL-10 is the a nucleotide sequence encoding the amino acid sequence of SEQ ID NO: 6, SEQ ID NO: 9, SEQ ID NO: 38, SEQ ID NO: 40, or SEQ ID NO: 42.

9. The pharmaceutical composition of claim 1, wherein the gene encoding IL-10 is the nucleotide sequence of SEQ ID NO: 7, SEQ ID NO: 8, SEQ ID NO: 10, SEQ ID NO: 14, SEQ ID NO: 39, SEQ ID NO: 41, or SEQ ID NO: 43.

10. The pharmaceutical composition of claim 1, wherein the gene encoding GDNF is a nucleotide sequence encoding the amino acid sequence of SEQ ID NO: 11, SEQ ID NO: 44, SEQ ID NO: 46, or SEQ ID NO: 48.

11. The pharmaceutical composition of claim 1, wherein the gene encoding GDNF is the nucleotide sequence of SEQ ID NO: 12, SEQ ID NO: 13, SEQ ID NO: 45, SEQ ID NO: 47 or SEQ ID NO: 49.

12. (canceled)

13. The pharmaceutical composition of claim 1, wherein the pharmaceutical composition further comprises a physiologically acceptable carrier.

14. The pharmaceutical composition of claim 1, wherein the pharmaceutical composition is an injection formulation.

15. A method for alleviating or treating pain, comprising administering the pharmaceutical composition of claim 1 to a subject in need thereof.

16. The method of claim 15, wherein the administering is administration via epidural injection or intrathecal injection.

17. The method of claim 15, wherein the pain is nociceptive pain, psychogenic pain, inflammatory pain, pathological pain, neuropathic pain, cancer pain, postoperative pain, trigeminal neuralgia pain, idiopathic pain, diabetic neuropathic pain, or migraine.

18. (canceled)

19. (canceled)
